(19) United States
(12) Patent Application Publication
(10) Pub. No.: US 2012/0237473 A1
(43) Pub. Date: Sep. 20, 2012

(54) Compositions And Methods For Cell Based Retinal Therapies

(75) Inventors: Anton Kolomeyer, Highland Park, NJ (US); Ilene Sugino, Madison, NJ (US); Marco Zarbin, Chatham, NJ (US)

(73) Assignee: UNIVERSITY OF MEDICINE AND DENTISTRY OF NEW JERSEY, Somerset, NJ (US)

(21) Appl. No.: 13/420,502

(22) Filed: Mar. 14, 2012

Related U.S. Application Data
(60) Provisional application No. 61/452,458, filed on Mar. 14, 2011.
(60) Provisional application No. 61/452,487, filed on Mar. 14, 2011.

(51) Int. Cl. A61K 38/18; A61K 38/17; A61K 38/30; C12N 5/071; A61P 27/06; C12Q 1/02; C12Q 1/32; G01N 33/566; A61K 38/19; A61P 27/02

(52) U.S. Cl. 424/85.1; 514/9.1; 514/9.6; 514/8.1; 514/8.4; 514/20.8; 514/8.7; 514/8.9; 514/9.5; 435/29; 435/26; 435/6.19; 435/325; 435/366

(57) ABSTRACT

The invention relates to pharmaceutical compositions comprising trophic factors, methods to decrease the degeneration of a retina, methods of treating ocular degenerative diseases and methods to select cells for transplantation.

### CROSS-REFERENCE TO RELATED APPLICATION

This application claims priority of U.S. Provisional Application No. 61/452,458 filed on Mar. 14, 2011 and U.S. Provisional Application No. 61/452,487 filed on Mar. 14, 2011, the disclosures of which are incorporated herein by reference in their entirety.

### FIELD OF THE INVENTION

The invention relates to pharmaceutical compositions comprising trophic factors, methods to decrease the degeneration of a retina, methods of treating ocular degenerative diseases and methods to select cells for transplantation.

### BACKGROUND OF THE INVENTION

The retinal pigment epithelium (RPE) is a monolayer of hexagonal, cuboidal, pigmented cells. It is critical for photoreceptor cell and choroid homeostasis. RPE cell degeneration results in abnormal photoreceptor morphology, choriocapillaris degeneration, and alteration of proper retinal function with eventual photoreceptor cell death. Maintenance of normal retinal physiology relies on a wide variety of RPE functions, including growth/trophic factor secretion. Trophic factors are endogenously produced substances (either proteins or steroid hormones) that bind to cell surface or nuclear receptors and generally function to promote cell proliferation, maturation, survival, and/or regeneration by activating a number of downstream pathways. The importance of growth/trophic factor support by RPE in the prevention of photoreceptor cell death was initially proposed in the study of rat chimeras and models of retinal degeneration, further substantiated by numerous other investigators.

Retinal degenerative diseases constitute the leading causes of blindness in the industrialized world. Age-related macular degeneration (AMD), the most prevalent of these, can be treated pharmacologically, although at this time the majority of patients do not recover lost vision. Furthermore, patients who suffer vision loss are infrequently able to recover any measure of it. Thus, there remains a need for practicable and efficacious therapies, including cell based therapies, for retinal degenerative diseases and their symptoms.

### SUMMARY OF THE INVENTION

The present invention relates to pharmaceutical compositions comprising biological trophic factors, methods to decrease the degeneration of a retina, methods of treating ocular degenerative diseases and methods to select cells for transplantation.

In one aspect, the invention provides a pharmaceutical composition that contains a pharmaceutically acceptable carrier and at least two biologically active trophic factors selected from the group of LIF, bFGF, HB-EGF, VEGF-A, NGF, BDNF, CNTF, PEDF, IGFBP-3, isoform 1 of semaphoring-3B, TGF-β and HGF. In certain embodiments, the selected biologically active trophic factors are HGF and PEDF. In another embodiment, the pharmaceutical composition contains LIF, bFGF, HB-EGF, VEGF-A, NGF, BDNF, CNTF, HGF and PEDF. In certain embodiments, the pharmaceutically acceptable carrier is capable of sustained release delivery of the trophic factors.

In a second aspect, the invention provides a method to decrease the degeneration of a retina associated with an ocular condition in a subject in need thereof by administrating to said subject an effective amount of the pharmaceutical composition that contains a pharmaceutically acceptable carrier and at least two biologically active trophic factors selected from the group of LIF, bFGF, HB-EGF, VEGF-A, NGF, BDNF, CNTF, PEDF, IGFBP-3, isoform 1 of semaphoring-3B, TGF-β and HGF. The pharmaceutical composition may be administered by intraocular administration. The ocular condition may be age-related macular degeneration, retinitis pigmentosa, glaucoma, optic atrophy, ocular inflammation, retinopathy, diabetic retinopathy, retinal ganglion cell dysfunction, and retinal detachment.

In a third aspect, the invention provides a method to select a group of cells that secrete factors that decrease the degeneration of a retina by performing the following steps: a) isolating medium from a group of cultured cells; b) culturing a degenerating animal retina with said isolated medium; c) determining the degeneration of the retina; and d) comparing the level of the degeneration of the retina to a standard; wherein the group of cells are selected if the isolated medium decreased the degeneration of the retina compared to the standard. The group of cells may be stem cells capable of differentiating to retinal pigment epithelial cells, or fetal retinal pigment epithelial cells. In certain embodiments the determining step is conducted by performing a cytotoxocity assay and/or an apoptotic assay. The standard may be the level of the degeneration of a retina in-non-conditioned media. In certain embodiments the cells are human cells. The group of cultured cells may be cultured in a solid support. Also, the degenerating retina may be cultured in a solid support. The animal retina may be a human or a porcine retina.

In a fourth aspect, the invention provides conditioned media isolated from fetal retinal pigment epithelial cells cultured in medium in a solid support, wherein said medium is isolated after said cells have been passaged from 2 to 6 passages and within 7 days of said passage.

In a fifth aspect, the invention provides a method of identifying cells for transplantation to a subject having an ocular condition by performing the following steps: a) isolating medium from a group of cultured cells; b) culturing a degenerating animal retina with said isolated medium; c) determining the degeneration of the retina; and d) comparing the level of the degeneration of the retina to a standard; wherein the group of cells are selected if said isolated medium decreased the degeneration of the retina compared to a standard. The group of cells may be human cells, or the group of cells may be stem cells isolated from the subject. The group of cells may be cultured in a solid support. Also, the degenerating retina may be cultured in a solid support. The animal retina may be a human or a porcine retina.

In a sixth aspect, the invention provides a kit for identifying an agent that decreases the degeneration of a retina, the kit contains an explant culture of full thickness animal retina. The retina may be a human or a porcine retina. The kit may further contain reagents for performing an assay for measuring retinal degradation.

### BRIEF DESCRIPTION OF THE DRAWINGS

FIG. 1 illustrates the effect of culture medium (CM) collected from adult and AMD eye-cup preparations on porcine retinal cytotoxicity.

FIG. 2 illustrates the effect of CM collected from adult and AMD eye-cup preparations on porcine retinal apoptosis.

FIG. 3 illustrates the effect of CM collected from adult and fetal eye-cup preparations on porcine retinal cytotoxicity.

FIG. 4 illustrates the effect of CM collected from adult and fetal eye-cup preparations on porcine retinal apoptosis.

FIG. 5 illustrates the effect of conditioned media (CM) collected from passage-2, day-7 and passage-6, day-7 cultured fetal RPE cells on porcine retinal cytotoxicity

FIG. 6 illustrates the effect of conditioned media (CM) collected from passage-2, day-7 and passage-6, day-7 cultured fetal RPE cells on porcine retinal apoptosis.

FIG. 7 illustrates the effect of conditioned media (CM) collected from passage-2, day-7 cultured fetal RPE cells and primary cultured adult RPE cells on porcine retinal cytotoxicity.

FIG. 8 illustrates the effect of conditioned media (CM) collected from passage-2, day-7 cultured fetal RPE cells and primary cultured adult RPE cells on porcine retinal apoptosis.

FIG. 9 illustrates the effect of heating and proteinase-K treatment on RPE-CM modulation of porcine retinal cytotoxicity (FIG. 9A) and apoptosis (FIG. 9B).

FIG. 10 illustrates the effect of RPE-CM concentration on porcine retinal cytotoxicity (FIG. 10A) and apoptosis (FIG. 10B).

### DETAILED DESCRIPTION OF THE INVENTION

### 1. Overview

The present invention relates to pharmaceutical compositions comprising biological trophic factors, methods to decrease the degeneration of a retina, methods of treating ocular degenerative diseases and methods to select cells for transplantation.

### 2. Definitions

The terms “polypeptide”, “peptide”, “protein”, and “protein fragment” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “biologically active fragment” is meant a fragment of a full-length parent polypeptide which fragment retains an activity of the parent polypeptide. As used herein, the term “biologically active fragment” includes deletion variants and small peptides, for example of at least 6, 7, 8, 9, 10, 11, 12, 13, 14, 15, 16, 17, 18, 19, 20, 21, 22, 23, 24, 25, 26, 27, 28, 29, 30, 35, 40, 45, 50 contiguous amino acid residues, which comprise an activity of the parent polypeptide. Peptides of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “medium” and “media” are used interchangeably

The term “solid support” refers to a culture vessel for cells that can be any shape and size including a well in multi-well tissue culture plate, or as large as a stirred tank bioreactor. For large-scale applications, the surface area for cell attachment can be increased by the use of microbeads or other substrates that can be suspended in a culture medium (e.g., plastic beads or polymers) or the like may be used, either coated or uncoated.

The term “trophic factor” and “growth factor” are used interchangeably and mean either proteins or steroid hormones, endogenously produced substances by a cell that bind to cell surface or nuclear receptors and generally function to promote cell proliferation, maturation, survival, and/or regeneration by activating a number of downstream pathways.

### 3. Pharmaceutical Compositions

The present invention provides pharmaceutical compositions comprising a pharmaceutically acceptable carrier and at least at least 2, 3, 4, 5, 6, 7, 8 or 9 biologically active trophic factors selected from the group consisting of LIF, bFGF, HB-EGF, VEGF-A, NGF, BDNF, CNTF, PEDF, igfbp-3, isoform 1 of semaphoring-3B, TGF-β and HGF, including biologically active fragments thereof. In a preferred embodiment the pharmaceutical composition comprises at least PEDF and HGF. In a further embodiment the composition comprises LIF, bFGF, HB-EGF, VEGF-A, NGF, BDNF, CNTF, HGF and PEDF. The following are the preferred concentration ranges for the factors based upon picograms (pg) per milliliter (ml): LIF is about 1.0-1200 pg/ml; bFGF is about 5.0-4900 pg/ml; HB-EGF is about 1.0-500 pg/ml; HGF is about 1.0-37000 pg/ml; VEGF-A is about 20-141000 pg/ml; NGF is about 1.0-260 pg/ml; BDNF is about 2.0-2200 pg/ml; CNTF is about 5.0-2600 pg/ml; and PEDF is about 129000-13700000 pg/ml.

The relative ratios of the trophic factors can be modified to improve retinal preservation. For example, a significantly lower relative ratio of IGFBP-3, semaphorin-3B, and TGF-β in the pharmaceutical composition could be of benefit to retinal preservation. A significantly higher relative ratio of HDGF, gelsolin, and PEDF in the pharmaceutical composition could be of benefit to retinal preservation. Using routine methods in the art, one with ordinary skill in the art can optimize the relative ratios of the trophic factors to decrease retinal degeneration.

The trophic factors may be derived from a cell or the medium isolated from a cultured cell. In a preferred embodiment the cell is cultured in a solid support. The cells may be cells that secrete any of the following trophic factors: LIF, bFGF, HB-EGF, VEGF-A, NGF, BDNF, CNTF, PEDF, igfbp-3, isoform 1 of semaphoring-3B, TGF-β and HGF. The cells may be fetal RPE cells, and preferably are human fetal RPE cells. The cells may be fetal RPE cells that are cultured in medium and a solid support, and the medium is collected after 2 to 6 passages, within 7 days of the passage. Most preferably the medium is collected from fetal RPE cells on the seventh day of the second passage. It is preferable that the medium collected is free from any cellular debris, for example centrifuging the media at 1000 rpm for 5 minutes.

The factors may also be produced by methods known in the art, for example by recombinant methods, or may be obtained commercially.

In a preferred embodiment, the concentration of the trophic factor is the concentration required to achieve greater than 50% receptor occupancy for the receptor for the factor. In a more preferred embodiment, the concentration of the trophic factor is the concentration required to achieve 90% or greater receptor occupancy for the receptor for the factor. In a further embodiment, the trophic factor is HGF, VEGF-A and/or PEDF, or any combination thereof.

Receptor occupancy is the calculated mean (±SEM) percent receptor occupancy for each trophic factor and its primary receptor. Biologically significant (vs. statistically significant) changes in trophic factor concentration should be associated with significant changes in relevant receptor occupancy. (Khodair M A, et. al. Invest Ophthalmol Vis Sci 2003; 44:4976-4988) (For example, if a trophic factor, L, concentration increases from [L]₁ to [L]₂, but [L]₁ already saturates the target receptor, then the change in concentration is not likely to be biologically significant, assuming that the trophic factor effect is mediated via the receptor in question.) The following assumptions are made: 1) receptor-ligand interactions occur according to simple mass action kinetics; 2) adaptation (e.g., endocytic receptor downregulation or ligand-induced receptor desensitization) is not being considered; 3) receptor occupancy directly results in receptor functionality; and 4) small changes in receptor occupancy might be significant provided that the ligand concentrations are below saturation. The magnitude of a biological response is directly proportional to the receptor-ligand complex concentration. Thus, increases in trophic factor concentration that lead to significant changes in receptor occupancy are expected to be biologically relevant. Mathematically, occupancy is defined as the proportion of the concentration of the receptor-ligand complex (i.e., bound receptor) divided by the total concentration of the receptor (i.e., the ligand-bound receptor plus the un-bound receptor) (equation 1). It is related to the dissociation constant (K_{D}), which is defined as the product of the concentrations of the free ligand and the free receptor concentration divided by the concentration of the receptor-ligand complex (equation 2). After rearranging the equations, occupancy equals the concentration of the ligand divided by the quantity, K_{D} plus the concentration of the ligand (equation 3). K_{D} values for each trophic factor receptor were identified through the PubMed search engine. Only trophic factor receptors specific to the retina, RPE, and the choroid were included. Potential biological activity was only assumed from the calculated changes in trophic factor receptor occupancies and did not mathematically factor into the calculations.

Occupancy=[RL]/[RL+R], Equation 1
where R—unbound receptor, L—ligand, and RL—receptor-ligand complex

Dissociation constant(K_{D})=[R][L]/[RL], Equation 2
where R—receptor, L—ligand, and RL—receptor-ligand complex

Receptor occupancy=[L]/[K_{D}+L], Equation 3
where L—ligand and K_{D}—dissociation constant

The trophic factors can be an isolated or purified protein. An “isolated” or “purified” protein refers to protein that has been separated from other proteins, lipids, and nucleic acids with which it is naturally associated. The polypeptide/protein can constitute at least 10% (i.e., any percentage between 10% and 100%, e.g., 20%, 30%, 40%, 50%, 60%, 70%, 80%, 85%, 90%, 95%, and 99%) by dry weight of the purified preparation. Purity can be measured by any appropriate standard method, for example, by column chromatography, polyacrylamide gel electrophoresis, or HPLC analysis. An isolated protein described in the invention can be purified from a natural source, produced by recombinant DNA techniques, or by chemical methods.

The trophic factors may also be pegylated to improve the half life and stability. The trophic factors may also be conjugated to biodegradable polymers to improve the stability of the factor, as well as for sustained release delivery of the pharmaceutical composition.

“Biodegradable” for the purposes of this invention means the ability of any biocompatible material to breakdown within the physiological environment of the eye by one or more physical, chemical, or cellular processes at a rate consistent with providing structural or pharmaceutical barriers (or both) at a therapeutic level controllable by selection of a polymer or mixture of polymers (also referred to as polymeric materials), including, but not limited to: polylactide polymers (PLA), copolymers of lactic and glycolic acids (PLGA), polylactic acid-polyethylene oxide copolymers, poly(ε-caprolactone-co-L-lactic acid (PCL-LA), glycine/PLA copolymers, PLA copolymers involving polyethylene oxides (PEO), acetylated polyvinyl alcohol (PVA)/polycaprolactone copolymers, hydroxybutyrate-hydroxyvalerate copolymers, polyesters such as, but not limited to, aspartic acid and different aliphatic diols, poly(alkylene tartrates) and their copolymers with polyurethanes, polyglutamates with various ester contents and with chemically or enzymatically degradable bonds, other biodegradable nonpeptidic polyamides, amino acid polymers, polyanhydride drug carriers such as, but not limited to, poly(sebacic acid) (PSA), aliphatic-aromatic homopolymers, and poly(anhydride-co-imides), poly(phosphoesters) by matrix or pendant delivery systems, poly(phosphazenes), poly(iminocarbonate), crosslinked poly(ortho ester), hydroxylated polyester-urethanes, or the like. The polymer can be a gel or hydrogel type polymer, PLA or PLGA polymer or mixtures or derivatives thereof.

The pharmaceutical composition comprising the trophic factors and pharmaceutically acceptable carrier, may be in the form of biodegradable polymeric implants, non-biodegradable polymeric implants, biodegradable polymeric microparticles, and combinations thereof. Implants may be in the form of rods, wafers, sheets, filaments, spheres, and the like. Particles are generally smaller than the implants disclosed herein, and may vary in shape. For example, certain embodiments of the present invention utilize substantially spherical particles. These particles may be in the form of microspheres. Other embodiments may utilize randomly configured particles, such as particles that have one or more flat or planar surfaces. The drug delivery system may comprise a population of such particles with a predetermined size distribution. For example, a major portion of the population may comprise particles having a desired diameter measurement. Additional sustained release delivery biodegradeable polymer, microparticle and implant formulations are described in U.S. Patent Publication No. 2012/0059060.

“Microsphere” and “microparticle” are used synonymously to refer to a small diameter or dimension (see below) device or element that is structured, sized, or otherwise configured to be administered subconjunctivally (i.e. sub-tenon), subretinally, or into the vitreous. Microspheres or microparticles includes particles, micro or nanospheres, small fragments, microparticles, nanoparticles, fine powders and the like comprising a biocompatible matrix encapsulating or incorporating the pharmaceutical composition. Microspheres are generally biocompatible with physiological conditions of an eye and do not cause significant adverse side effects. Microspheres administered intraocular can be used safely without disrupting vision of the eye. Microspheres have a maximum dimension, such as diameter or length, less than 1 mm. For example, microparticles can have a maximum dimension less than about 500 μm. Microspheres can also have a maximum dimension no greater than about 200 μm, or may have a maximum dimension from about 30 μm to about 50 μm, among other sizes.

The pharmaceutical composition may further comprise antibiotics. Examples of antibiotics include without limitation, cefazolin, cephradine, cefaclor, cephapirin, ceftizoxime, cefoperazone, cefotetan, cefutoxime, cefotaxime, cefadroxil, ceftazidime, cephalexin, cephalothin, cefamandole, cefoxitin, cefonicid, ceforanide, ceftriaxone, cefadroxil, cephradine, cefuroxime, ampicillin, amoxicillin, cyclacillin, ampicillin, penicillin G, penicillin V potassium, piperacillin, oxacillin, bacampicillin, cloxacillin, ticarcillin, azlocillin, carbenicillin, methicillin, nafcillin, erythromycin, tetracycline, doxycycline, minocycline, aztreonam, chloramphenicol, ciprofloxacin hydrochloride, clindamycin, metronidazole, gentamicin, lincomycin, tobramycin, vancomycin, polymyxin B sulfate, colistimethate, colistin, azithromycin, augmentin, sulfamethoxazole, trimethoprim, derivatives thereof, and the like and mixtures thereof.

### 4. Methods to Decrease the Degeneration of a Retina

The invention provides a method to decrease the degeneration of a retina associated with an ocular condition in a subject in need thereof comprising administration of a pharmaceutical composition of the invention to the subject in an amount effective to decrease degeneration of the retina.

To administer the pharmaceutical composition to a subject, it is preferable to formulate the trophic factors in a composition comprising one or more pharmaceutically acceptable carriers. The phrase “pharmaceutically acceptable” refers to molecular entities and compositions that do not produce allergic, or other adverse reactions when administered using routes well-known in the art. “Pharmaceutically acceptable carriers” include any and all clinically useful solvents, dispersion media, coatings, antibacterial and antifungal agents, isotonic and absorption delaying agents and the like.

Examples of pharmaceutically acceptable carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present invention.

The dose of the pharmaceutical composition of the present invention is determined according to the age, body weight, general health condition, sex, diet, administration time, administration method, clearance rate, and the level of disease for which patients are undergoing treatments at that time, or further in consideration of other factors. While the daily dose of the compound of the present invention varies depending on the condition and body weight of patient, the kind of the compound, administration route and the like, in regards to intraocular administration, for example, 0.01 to 100 mg/patient/day.

The present invention provides pharmaceutical compositions that can be administered as depot injectable formulations, for example a biodegradable polymer hydrogel. The present invention also provides for depot injectable formulations that are prepared by entrapping the trophic factors and pharmaceutically acceptable carrier in liposomes or microemulsions which are compatible with body tissue. When the trophic factors of the present invention are administered as pharmaceuticals, to a subject, they can be given per se or as a pharmaceutical composition containing, for example, 0.1 to 99.5% (more preferably, 0.5 to 90%) of the trophic factors in combination with a pharmaceutically acceptable carrier.

The present compositions may be placed into the interior of an eye using a syringe, a needle, a cannula, a catheter, a pressure applicator, and the like.

Generally the ocular condition is related to a damaged retina and/or retinal degenerative disease. Examples of ocular conditions include without limitation MACULOPATHIES/RETINAL DEGENERATION: Non-Exudative Age Related Macular Degeneration (ARMD), Exudative Age Related Macular Degeneration (ARMD), wet macular degeneration, Choroidal Neovascularization, Diabetic Retinopathy, Acute Macular Neuroretinopathy, Central Serous Chorioretinopathy, Cystoid Macular Edema, Diabetic Macular Edema. UVEITIS/RETINITIS/CHOROIDITIS: Acute Multifocal Placoid Pigment Epitheliopathy, Behcet's Disease, Birdshot Retinochoroidopathy, Infectious (Syphilis, Lyme, Tuberculosis, Toxoplasmosis), Intermediate Uveitis (Pars Planitis), Multifocal Choroiditis, Multiple Evanescent White Dot Syndrome (MEWDS), Ocular Sarcoidosis, Posterior Scleritis, Serpignous Choroiditis, Subretinal Fibrosis and Uveitis Syndrome, Vogt-Koyanagi-Harada Syndrome. VASCULAR DISEASES/EXUDATIVE DISEASES: Retinal Arterial Occlusive Disease, Central Retinal Vein Occlusion, Disseminated Intravascular Coagulopathy, Branch Retinal Vein Occlusion, Hypertensive Fundus Changes, Ocular Ischemic Syndrome, Retinal Arterial Microaneurysms, Coat's Disease, Parafoveal Telangiectasis, Hemi-Retinal Vein Occlusion, Papillophlebitis, Central Retinal Artery Occlusion, Branch Retinal Artery Occlusion, Carotid Artery Disease (CAD), Frosted Branch Angitis, Sickle Cell Retinopathy and other Hemoglobinopathies, Angioid Streaks, Familial Exudative Vitreoretinopathy, Eales Disease. TRAUMATIC/SURGICAL: Sympathetic Ophthalmia, Uveitic Retinal Disease, Retinal Detachment, Trauma, Laser, PDT, Photocoagulation, Hypoperfusion During Surgery, Radiation Retinopathy, Bone Marrow Transplant Retinopathy. PROLIFERATIVE DISORDERS: Proliferative Vitreal Retinopathy and Epiretinal Membranes, Proliferative Diabetic Retinopathy. INFECTIOUS DISORDERS: Ocular Histoplasmosis, Ocular Toxocariasis, Presumed Ocular Histoplasmosis Syndrome (POHS), Endophthalmitis, Toxoplasmosis, Retinal Diseases Associated with HIV Infection, Choroidal Disease Associated with HIV Infection, Uveitic Disease Associated with HIV Infection, Viral Retinitis, Acute Retinal Necrosis, Progressive Outer Retinal Necrosis, Fungal Retinal Diseases, Ocular Syphilis, Ocular Tuberculosis, Diffuse Unilateral Subacute Neuroretinitis, Myiasis. GENETIC DISORDERS: Retinitis Pigmentosa, Systemic Disorders with Accosiated Retinal Dystrophies, Congenital Stationary Night Blindness, Cone Dystrophies, Stargardt's Disease and Fundus Flavimaculatus, Best's Disease, Pattern Dystrophy of the Retinal Pigmented Epithelium, X-Linked Retinoschisis, Sorsby's Fundus Dystrophy, Benign Concentric Maculopathy, Bietti's Crystalline Dystrophy, pseudoxanthoma elasticum. RETINAL TEARS/HOLES: Retinal Detachment, Macular Hole, Giant Retinal Tear. TUMORS: Retinal Disease Associated with Tumors, Congenital Hypertrophy of the RPE, Posterior Uveal Melanoma, Choroidal Hemangioma, Choroidal Osteoma, Choroidal Metastasis, Combined Hamartoma of the Retina and Retinal Pigmented Epithelium, Retinoblastoma, Vasoproliferative Tumors of the Ocular Fundus, Retinal Astrocytoma, Intraocular Lymphoid Tumors. MISCELLANEOUS: Punctate Inner Choroidopathy, Acute Posterior Multifocal Placoid Pigment Epitheliopathy, Myopic Retinal Degeneration, Acute Retinal Pigment Epithelitis, Stroke and the like.

### 5. Methods and Kits for Selecting Cells

In another embodiment of the present invention, retinal tissue provides a screening assay to evaluate the potential suitability of candidate cell lines to improve the viability of degenerating photoreceptor cells and/or the remainder of the neural retina. This approach may be applied to cell-based therapy of ocular and CNS degenerative diseases, retinal detachment, glaucoma, and/or stroke. The in vitro retina assays of the present invention are also useful for pharmacological screening of drug therapies to prevent retinal degeneration.

In one embodiment, the present invention provides methods to select cells that secrete factors that decrease the degeneration of a retina. The present method comprises the following steps: (a) isolating medium from a group of cells cultured in a solid support; (b) culturing a degenerating retina in a solid support with said isolated medium; (c) determining the degeneration of the retina; and (d) comparing level of the degeneration of the retina to a standard, and selecting the group of cells if the isolated medium decreased the degeneration of the retina compared to the standard. The degenerating retina may be an in vitro explant culture of full thickness animal retina. In one embodiment, the degenerating retina is a full thickness porcine retina, which has been demonstrated herein to provide a surrogate for human retina. In another embodiment, the degenerating retina is a full thickness human retina. In a preferred embodiment, the standard can be the level of the degeneration of a retina cultured in non-conditioned medium In a further embodiment, a small molecule may be added to Step (a) to screen for new drug therapies or substituted for the isolated medium is Step (a). In a further embodiment, the cells that are selected may be used for transplantation in a subject with an ocular condition.

The group of cells may be RPE cells, stem cells capable of differentiating to RPE cells, and fetal RPE cells. The cells may be RPE cells or stem cells isolated from a subject. The stem cells may be pluripotent cells that are programmed to secrete trophic factors: bFGF, HB-EGF, VEGF-A, NGF, BDNF, CNTF, HGF and PEDF; as well as be efficacious at reducing retinal cytotoxicity and apoptosis of a retina. Methods are known in the art to characterize the trophic factor secretion profile of a group of cells, such as the isobaric tag for relative and absolute quantification (iTRAQ) multiplex global protein analysis. This assay allows one with ordinary skill in the art to simultaneously indentify and quantify proteins from different sources in one experiment. (Zieske L R. J Exp Bot 2006; 57:1501-1508 and Liu T, et al. J Proteome Res 2007; 6:2565-2575.) In a preferred embodiment, the cells are human cells.

In the foregoing methods, the level of degeneration of the retina can be determined by biochemical methods and/or morphological/histological methods. Biochemical methods such as a cytotoxicity assay and/or an apoptotic assay can be performed on the degenerating retina. A cytotoxicity assay such as the lactate dehydrogenase (LDH) in vitro toxicology assay can be used to determine the effects of the culture medium on the retinal membrane integrity, thus concluding the amount of viable cells after certain time points. An apoptotic assay such as a cell-death detection ELISA kit can be used to quantify the effects of the culture medium on the amount of retinal DNA fragmentation, which is useful for differentiating apoptosis from necrosis. One with ordinary skill in the art can determine the parameters of the assays, including the type of retina, amount of the candidate group of cells, and the time points for collection of the retina sample to be assayed for cytotxocity and apoptosis to determine the degeneration of the retina. It is preferable to collect samples of the degenerating retina of culture with the culture medium isolated from the group of cells after 1, 6, 24 and/or 48 hours. Multi-well plates can be used for more than one sample.

In other embodiments, morphological/histological methods include outer nuclear layer (ONL), thickness, number of nuclear rows in the ONL, and, degree of photoreceptor axon and terminal retraction into the ONL. Explants can be fixed and vibratomed. Sections of the retina can be stained with Propidium Iodide (nuclear layers) and synaptic vesicle protein-2 (SV2; photoreceptor axon terminals) then observed. The thickness and stratification of the outer nuclear layer (ONL) can also be measured. The area of synaptic vesicle protein-2 (SV2) within the ONL represents the degree of photoreceptor axon/terminal retraction that can be quantified. The less photoreceptor terminal/axon retraction compared to an untreated control retina correlates with a decrease in retinal degeneration. Also a thicker ONL correlates with a decrease in retinal degeneration. One with ordinary skill in the art can utilize routine methods and instrumentation known in the art to observe the morphological differences of retina degeneration. It is preferable to collect samples of the degenerating retina of culture with the culture medium isolated from the group of cells after 1, 6, 24 and/or 48 hours. Multi-well plates can be used for more than one sample.

The cells identified by the foregoing methods are useful for transplantation to various target sites within a subject's eye. For example, the cells may be transplanted to the subretinal space of the eye, which is the normal anatomical location of the RPE (between the photoreceptor outer segments and the choroids). In addition, dependant upon migratory ability and/or positive paracrine effects of the cells, transplantation into additional ocular compartments can be considered including the vitreal space, the inner or outer retina, the retinal periphery and within the choroids.

Transplantation may be performed by various techniques known in the art. Methods for performing RPE transplants are described in, for example, U.S. Pat. Nos. 5,962,027, 6,045,791, and 5,941,250 and in Eye Graefes Arch Clin Exp Opthalmol March 1997; 235(3):149-58; Biochem Biophys Res Commun Feb. 24, 2000; 268(3): 842-6; Opthalmic Surg February 1991; 22(2): 102-8. Methods for performing corneal transplants are described in, for example, U.S. Pat. No. 5,755,785, and in Eye 1995; 9 (Pt 6 Su):6-12; Curr Opin Opthalmol August 1992; 3 (4): 473-81; Ophthalmic Surg Lasers April 1998; 29 (4): 305-8; Opthalmology April 2000; 107 (4): 719-24; and Jpn J Opthalmol November-December 1999; 43(6): 502-8. If mainly paracrine effects are to be utilized, cells may also be delivered and maintained in the eye encapsulated within a semi-permeable container, which will also decrease exposure of the cells to the host immune system (Neurotech USA CNTF delivery system; PNAS Mar. 7, 2006 vol. 103(10) 3896-3901).

Transplantation may also be performed via pars pana vitrectomy surgery followed by delivery of the cells through a small retinal opening into the sub-retinal space or by direct injection. Alternatively, cells may be delivered into the subretinal space via a transscleral, transchoroidal approach. In addition, direct transscleral injection into the vitreal space or delivery to the anterior retinal periphery in proximity to the ciliary body can be performed.

The cells may be transplanted in various forms. For example, the cells may be introduced into the target site in the form of a cell suspension, or adhered onto a matrix, extracellular matrix or substrate such as a biodegradable polymer or a combination. The cells may also be transplanted together (co-transplantation) with other retinal cells, such as with photoreceptors or other RPE cells.

### 5. Cells and Culture Medium

The present invention provides cells that secrete trophic factors that decrease the degeneration of a retina. The cells secrete one or more trophic factors selected from HGF, bFGF, HB-EGF, VEGF-A, NGF, BDNF, CNTF, HGF and PEDF. The cells may be retinal pigment epithelial cells, fetal retinal pigment epithelial cells, and/or stem cells capable of differentiating to retinal pigment epithelial cells. The cells are preferably human cells. The cells may be fetal RPE cells that are cultured in medium and a solid support, and collected after 2 to 6 passages, within 7 days of the passage. Most preferably the fetal RPE cells are collected after 2 passages and collected on the seventh day of the passage. Methods are know in the art, such as the isobaric tag for relative and absolute quantification (iTRAQ) multiplex global protein analysis to determine the trophic factor secretion profile of the group of cells. (Zieske L R. J Exp Bot 2006; 57:1501-1508 and Liu T, et al. J Proteome Res 2007; 6:2565-2575.)

The present invention provides culture medium, isolated from cells, comprising trophic factors that decrease the degeneration of a retina. In a preferred embodiment the trophic factors are one or more of LIF, bFGF, HB-EGF, VEGF-A, NGF, BDNF, CNTF, PEDF, igfbp-3, isoform 1 of semaphoring-3B, TGF-β and HGF. The culture medium is more efficacious at reducing retinal cytotoxicity and apoptosis of a degenerating retina compared to unconditioned culture medium. The cells may be fetal RPE cells that are cultured in medium and a solid support, and the medium is collected after 2 to 6 passages, within 7 days of the passage. Most preferably the medium is collected from fetal RPE cells on the seventh day of the second passage. It is preferable that the medium collected from the cells is free from any cellular debris, for example centrifuging the media at 1000 rpm for 5 minutes.

The invention further provides a kit for identifying an agent that decreases degeneration of a retina. The kit comprises an explant culture of full thickness animal retina. The retina is preferably a human or porcine retina. The kit may further comprise reagents for performing an assay for measuring retinal degradation.

### EXAMPLES

The invention now being generally described, it will be more readily understood by reference to the following examples, which are included merely for purposes of illustration of certain aspects and embodiments of the present invention, and are not intended to limit the invention.

### Example 1

### Methods and Materials

Human Donor Eye Tissue

Eyes from non-AMD and AMD Caucasian donors of 55 years of age or older were obtained through various eye banks or suppliers (National Disease Resource Interchange, Inc., Philadelphia, Pa.; Vision Share, Apex, N.C.; Midwest Eye banks, Ann Arbor, Mich.; Tampa Lions Eye Institute for Transplant & Research, Tampa, Fla.; San Diego Eye bank, San Diego, Calif.) in the United States and Canada. The tissue acceptance criteria included: 1) no recent history of chemotherapy or radiation to the head; 2) not on a ventilator prior to death; 3) up to seven hours from death to enucleation with eyes preserved in a moist chamber and stored on ice immediately after removal; 4) no more than 48 hours from death to receipt; and 5) intact, bright, not opaque, orange-colored RPE monolayer as visualized through a dissecting microscope. Fetal eyes (17-22 weeks gestation) were obtained through Advanced Bioscience Resources, Inc (ABR; Alameda, Calif.). By these gestational ages, the RPE cell monolayer is formed completely. Upon arrival, eyes were cleaned of extraneous tissue, dipped in betadine solution (10% Povidone-iodine, The Purdue Frederique Company, Stamford, Conn.) which was immediately rinsed off with balanced salt solution, and washed twice for 10 minutes at 4° C. in Dulbecco's Modified Eagle Medium (DMEM with one g/l glucose, L-glutamine and sodium pyruvate, containing 3.7 g/L sodium bicarbonate) (Cellgro-Mediatech Inc., Manasses, Va.) supplemented with 250 μg/ml amphotericin B (Gibco-Invitrogen, Carlsbad, Calif.). Anterior segment and neural retina were removed subsequently, exposing the posterior segment, which contains the RPE, Bruch's membrane, choroid, and sclera posterior to the equator. The latter tissue is referred to as ‘eye-cup’.

TABLE 1
Adult donor eye information.

| Eye- | | | D-P | D-R |
|---|---|---|---|---|
| cup No. | A/G/E | Ocular Pathology | (h:m) | (h:m) |
| 1 | 92/M/C | a: none; b: hard macular drusen | 2:44 | 45:45 |
| 2 | 77/F/C | a: hard macular and peripheral drusen; | 1:57 | 26:25 |
| | | b: perimacular drusen | | |
| 3 | 73/M/C | a, b: posterior pole RPE clouding | 3:15 | 42:00 |
| 4 | 78/M/C | a: none; b: posterior pole RPE | 3:09 | 27:30 |
| | | hyperpigmentation | | |
| 5 | 89/M/C | a, b: peripheral chorioretinal atrophy | 3:39 | 36:15 |
| 6 | 69/M/C | a, b: posterior pole RPE clouding | 5:39 | 38:00 |
| 7 | 83/M/C | a, b: none | 2:09 | 35:55 |
| 8 | 71/M/C | a: RPE-choroid hyperpigmentation; | 5:21 | 44:25 |
| | | b: none | | |
| 9 | 82/M/C | a: none; b: small, peripapillary RPE | 5:15 | 33:00 |
| | | hyperpigmentation | | |
| 10 | 77/M/C | a, b: none | 2:21 | 41:11 |
| 11 | 69/F/C | a, b: none | 3:45 | 45:40 |
| 12 | 101/F/C | a, b: peripheral chorioretinal atrophy | 2:48 | 26:20 |

Abbreviations: No., number; A/G/E, age/gender/ethnicity; M, male; F, female; C, Caucasian; a and b, randomly assigned identity of fellow eyes; D-P, death to preservation; D-R, death to receipt; h:m, hours:minutes

TABLE 2
AMD eye donor information.

| Eye- | | | D-P | D-R |
|---|---|---|---|---|
| cup No. | A/G/E | Ocular Pathology | (h:m) | (h:m) |
| 1 | 86/F/C | a, b: soft, confluent macular drusen | 2:40 | 38:30 |
| | | and hard peripheral drusen | | |
| 2 | 77/F/C | a, b: small, soft macular drusen | 5:30 | 41:30 |
| 3 | 78/M/C | a, b: hard and soft macular and | 2:30 | 29:00 |
| | | perimacular drusen | | |
| 4 | 74/M/C | a: small retinal adhesions; | 6:25 | 46:00 |
| | | b: hard macular drusen | | |
| 5A | 93/F/C | a: macular adhesion with scar | 4:30 | 26:10 |
| 6 | 84/F/C | a, b: macular membrane with RPE | 3:40 | 46:20 |
| | | hyperpigmentation, and intermediate | | |
| | | macular and perimacular drusen | | |
| 7 | 86/M/C | a: large macular scar and circular, | 5:58 | 20:52 |
| | | perimacular RPE defect; b: hard and | | |
| | | soft macular and perimacular | | |
| | | drusen with RPE hyperpigmentation | | |
| 8 | 82/M/C | a, b: soft, confluent macular drusen | 5:00 | 40:20 |
| | | and peripapillary retinal adhesions | | |
| 9 | 86/F/C | a: hard and soft macular and peripheral | 4:45 | 25:25 |
| | | drusen and speckled RPE with | | |
| | | peripheral choroidal hyperpigmentation; | | |
| | | b: same as A plus macular drusen | | |
| | | associated RPE hyperpigmentation | | |
| 10 | 89/F/C | a, b: hard and soft macular drusen | 2:55 | 42:30 |
| | | with RPE hyperpigmentation, a | | |
| | | small macular membrane, and hard | | |
| | | peripheral drusen with RPE | | |
| | | hyperpigmentation | | |

Abbreviations: No., number; A/G/E, age/gender/ethnicity; M, male; F, female; C, Caucasian; a and b, randomly assigned identity of fellow eyes; D-P, death to preservation; D-R, death to receipt; h:m, hours:minutes

TABLE 3
Fetal eye donor information.

| Eye-cup | Gestational | | |
|---|---|---|---|
| No. | age (weeks) | Gender | Ethnicity |
| 1 | 19 | M | n/a |
| 2 | 18 | M | n/a |
| 3 | 20 | n/a | n/a |
| 4 | 20 | n/a | n/a |
| 5 | 20 | n/a | n/a |
| 6 | 20 | n/a | n/a |
| 7 | 18 | n/a | n/a |
| 8 | 20 | n/a | n/a |
| 9 | 21 | M | Caucasian |
| 10 | 22 | M | Caucasian |
| 11 | 20 | F | n/a |
| 12 | 18 | F | n/a |
| 13 | 22 | F | n/a |
| 14 | 17 | M | n/a |
| 15 | 19 | n/a | n/a |
| 16 | 22 | M | n/a |
| 17 | 18 | F | Caucasian |
| 18 | 17 | n/a | n/a |
| 19 | 19 | n/a | n/a |
| 20 | 17 | n/a | n/a |
| 21 | 19 | M | n/a |
| 22 | 21 | n/a | n/a |
| 23 | 21 | n/a | n/a |

Abbreviations: No., number; M, male; F, female; n/a, not available.
Note:
All fetal eyes were received within 24 hours post-harvest. Exact harvesting times were not provided by the eye banks due to patient confidentiality. None of these eyes had any discernible pathology.

Conditioned Medium (CM) Collection

Donor eyes: Eye-cups of non-AMD (‘adult’; n=12; mean age, 88.1 years), AMD (n=10; mean age, 83.0 years), and fetal (n=23; mean age, 19.6 weeks gestation) eyes were cleaned of extraneous tissue and washed in betadine (Tables 1-3). These preparations were filled with two ml (adult eye-cups) and ˜200 μl (fetal eye-cups; volume depending on gestational age) of DMEM and incubated for six hours at 37° C., 10% CO₂. The resultant CM (adult-CM, AMD-CM, and fetal-CM, respectively) was collected, centrifuged at 1000 rpm for five minutes (Model 5415c, Eppendorf, Hauppauge, N.Y.) to remove cellular debris, and supernatant was frozen at −80° C. (Bio Freezer, Form a Scientific, Evanston, Ill.).

Bruch's Membrane-Choroid-Sclera (BrM-C-S):

RPE cells were removed gently from adult (n=5; mean age, 86.2 years) and fetal (n=16; mean age, 19.6 weeks gestation) eye-cups. Bruch's membrane-choroid-sclera (BrM-C-S) eye-cups were washed twice with 200 μl (fetal) or two ml (adult) of DPBS (Cellgro-Mediatech), filled with 200 μl (fetal) and one ml (adult; keeping the medium level below the choroid-sclera separation plane created by mechanical RPE removal) of DMEM, and incubated for six hours at 37° C., 10% CO₂. The resultant BrM-C-S-CM was collected, centrifuged, and frozen. BrM-C-S trophic factor levels were expressed as pg/μg of BrM-C-S protein and calculated relative to the levels of trophic factors in adult- and fetal-CM. Additionally, the expression of bestrophin, an RPE-specific differentiation marker, was quantified by real-time PCR in order to determine whether there was RPE cell contamination of the BrM-C-S mRNA samples.

Trophic Factor Quantification

All preparations of CM were analyzed (in duplicate) via multiplex ELISA (Aushon Biosystems, Woburn, Mass.) for trophic factors (Table 4). These candidate factors were selected based on a literature search using the following criteria: 1) secretion by RPE cells and 2) preservation of photoreceptors and/or the retina. DMEM was analyzed for the same factors as a control for non-specific binding. This medium was quantified in duplicate on three separate occasions to ensure method reliability. CM from eye-cups of donor eyes as well as BrM-C-S were corrected for these values. CM from each eye was collected and analyzed separately. Values from each pair of eyes were averaged. An overall mean±SEM for each trophic factor was calculated and expressed as picograms of trophic factor per microgram of protein. Trophic factor detection frequencies, defined as the number of times a trophic factor was successfully identified by multiplex ELISA divided by the total number of CM samples analyzed, were calculated. Only trophic factors with a detection frequency ≧85% in adult, AMD, and fetal eye-cups were selected for further analysis.

TABLE 4
Candidate retinal and photoreceptor-preserving trophic
factors secreted by RPE cells. ^{14, 16, 36, 37, 54, 55}

| | MW | |
|---|---|---|
| Trophic factor | (kDa) | Biological Effect* |
| Brain-derived neurotrophic factor | 14 | Neurotrophic |
| (BDNF) | | |
| Ciliary neurotrophic factor (CNTF) | 24 | Neurotrophic |
| Epidermal growth factor (EGF) | 6 | Photoreceptor rescue |
| Basic fibroblast growth factor (bFGF) | 18 | Pro-angiogenic, |
| | | photoreceptor rescue |
| Glial-derived neurotrophic factor | 24 | Neurotrophic |
| (GDNF) | | |
| Heparin-binding epidermal growth | 23 | RPE proliferation, |
| factor (HB-EGF) | | VEGF-A secretion |
| Hepatocyte growth factor (HGF) | 83 | RPE survival, |
| | | neuroprotective |
| Interleukin-1 beta (IL-1β) | 31 | Photoreceptor survival |
| Nerve growth factor (NGF) | 13 | Neurotrophic, inflammation |
| Neurotrophin-3 (NT3) | 30 | Neurotrophic |
| Pigment epithelium-derived factor | 46 | Anti-angiogenic, |
| (PEDF) | | neurotrophic |
| Vascular endothelium growth factor- | 43 | Pro-angiogenic, |
| A (VEGF-A) | | photoreceptor development |
| Leukemia inhibitory factor (LIF) | 20 | Photoreceptor rescue, |
| | | RPE survival |
| Tumor necrosis factor-alpha | 26 | Photoreceptor rescue |
| (TNF-α) | | |

Abbreviations: MW, molecular weight;
*Only trophic effects are listed.

Protein Quantification

Protein was isolated from donor eye-cup RPE cells after the 6-hour CM collection period. After the eye-cups were washed twice with ice-cold DPBS, 200 μl of 1× lysis buffer (10 mM Tris, 500 μM EDTA, 75 mM NaCl, 0.5% Triton X-100, 5% glycerol, and 1% 100× protease inhibitor cocktail (Pierce-Thermo Fischer Scientific, Rockford, Ill.) prepared in dH₂O) was added. RPE cells were gently brushed off from the choroid into the lysis buffer, triturated on ice, sonicated three times for 10 seconds at 4° C. (Branson Sonifier 250; VWR Scientific, West Chester, Pa.), and centrifuged for 10 minutes at 10000 rpm, 4° C. Protein was also isolated from BrM-C-S eye-cups. The whole eye-cup (BrM-C-S tissue) was completely homogenized in 500 μl of 1× lysis buffer using a hard tissue disposable rotor stator generator probe (Omni TH; Omni International, Marietta, Ga.) after which the homogenate was centrifuged for 10 minutes at 10000 rpm, 4° C. The lysates were collected and frozen at −80° C. Protein was quantified using the Bradford reagent (Sigma-Aldrich, St. Louis, Mo.) according to manufacturer's instructions.

Real-Time Polymerase Chain Reaction (Real-Time PCR)

RNA was isolated from RPE cells of eight adult (average age, 84.0 years), five AMD (average age, 86.6 years), and 16 fetal (average age, 20.1 weeks gestation) eye-cups after the 6-hour CM collection period. After the eye-cups were washed twice with ice-cold DPBS, ˜80 μl (fetal eye-cups; depending on gestational age) or 200 μl (adult eye-cups) of RNeasy lysis buffer (RLT, Qiagen RNA Mini Kit, Valencia, Calif.) was added. RPE cells were gently brushed off from the choroid into the lysis buffer and homogenized by running the lysate through a shredder column (QIAshredder, Qiagen Inc., Valencia, Calif.). RNA was washed, bound, and eluted according to manufacturer's instructions (RNeasy Mini Kit, Qiagen). One μl of the eluted mRNA was used for quantification using a spectrophotometer (Nanodrop-1000, Thermo Fisher Scientific, Waltham, Mass.). The RT-PCR reaction, consisting of 600 ng of mRNA mixed with a high capacity cDNA reverse transcription kit (10×RT Buffer, 100 mM 25×dNTP mix, 10×RT Random Primers, MultiScribe Reverse Transcriptase, RNase inhibitor and nuclease-free dH₂O) (Applied Biosystems, Foster City, Calif.), was performed in a thermocycler (MJ Mini Personal Thermo Cycler, Bio-Rad) under the following conditions: 25° C. for 10 minutes, 37° C. for 120 minutes, 85° C. for five seconds, and cooled to 4° C. Real-time PCR for each trophic factor was done using 1 μl of cDNA, 1.25 μl of 20× TaqMan real-time PCR primers (proprietary sequence, Applied Bioscience), 12.5 μl of 2× TaqMan Universal PCR Master Mix (Applied Bioscience), 0.2 μl of 20 mg/ml BSA (Sigma-Aldrich), and 1.05 μl of dH₂O on a real time PCR system (Model 7500, Applied Bioscience) under the following conditions: 50° C. for two minutes, 95° C. for 10 minutes, 45 cycles of 15 seconds at 95° C., 60° C. for one minute, and held at 60° C. 18S rRNA transcript served as an endogenous control while Bestrophin served as a reference transcript. Trophic factors were then expressed in relation to the arbitrarily-chosen epidermal growth factor (EGF)_{adult} transcript, which was assigned a value of one. The corrections for the varying amounts of gene expression found in adult, AMD, and fetal samples were done according to the ΔΔCt method.³⁰ The results were calculated for each group of samples as mean±SEM of the level of EGF_{adult}.

Porcine Retina

Porcine Eye Tissue:

Eyes from 5- to 9-month, 150-230 lb, male and female American Yorkshire pigs were obtained from a local abattoir within three hours of enucleation (transported on ice). Porcine eyes were prepared for dissection by the method outlined previously for human donor eyes. Anterior segment and vitreous were removed, leaving the neural retina in the eye-cup. Six millimeter trephine blades (Storz Ophthalmic-Bausch and Lomb, Manchester, Mo.) were used to isolate equatorial, full-thickness retina tissue explants (avoiding the peripapillary region) by separating it from the RPE-BrM-C-S. These explants were randomly assigned to different culture conditions in order to negate the potential effects of selection bias and variability in retinal thickness. (Khodair M A, et al. Invest Ophthalmol Vis Sci 2003; 44:4976-4988.)

Retinal Cytotoxicity:

The lactate dehydrogenase (LDH) in vitro toxicology assay (TOX-7; Sigma-Aldrich) was used to assess the effects of various CM on retinal membrane integrity. Media from retinal explants collected at 1, 6, 24, and 48 hours of culture was centrifuged for five minutes at 1000 rpm to remove cellular debris. The supernatant was frozen at −20° C. and processed as per manufacturer's instructions. Colorimetric absorbances were assessed by a microplate reader (ELx800, BioTek, Winooski, Vt.) at 490 nm. The data are expressed relative to the 1-hour levels.

Retinal Apoptosis:

A cell death detection ELISA assay kit (Roche Diagnostics, Piscataway, N.J.) was used to quantify the effects of various CM on the amount of retinal DNA fragmentation. After 1, 6, 24, or 48 hours of culture, the explants were homogenized in 200 μl of provided lysis buffer by trituration, allowed to react for 30 minutes at room temperature, centrifuged for five minutes at 1000 rpm, 4° C., and the supernatant was frozen at −20° C. The specimens were processed as per manufacturer's instructions. Absorbances were measured at 405 nm with reference wavelength at 490 nm (ELx800, BioTek). A DNA-histone complex (included) served as the positive control. The data are expressed relative to the 1-hour levels.

Trophic Factor Receptor Occupancy

In order to relate the differences in the measured concentrations of secreted trophic factors in various CM to potential biological activity (defined as the ability of the CM to improve the survival of degenerating porcine retina), we calculated mean (±SEM) percent receptor occupancy for each trophic factor and its primary receptor. (For example, if a trophic factor, L, concentration increases from [L]₁ to [L]₂, but [L]₁ already saturates the target receptor, then the change in concentration is not likely to be biologically significant, assuming that the trophic factor effect is mediated via the receptor in question.) The following assumptions are made in this model: 1) receptor-ligand interactions occur according to simple mass action kinetics; 2) adaptation (e.g., endocytic receptor down regulation or ligand-induced receptor desensitization) is not being considered; 3) receptor occupancy directly results in receptor functionality; and 4) small changes in receptor occupancy might be significant provided that the ligand concentrations are below saturation. These assumptions may not apply for all trophic factors in complex systems such as the full-thickness retina. Adult-CM was used as a relative control for AMD- and fetal-CM. The basic premise of occupancy theory is that the magnitude of a biological response is directly proportional to the receptor-ligand complex concentration. Thus, increases in trophic factor concentration that lead to significant changes in receptor occupancy might be expected to be biologically relevant. Mathematically, occupancy is defined as the proportion of the concentration of the receptor-ligand complex (i.e., bound receptor) divided by the total concentration of the receptor (i.e., the ligand-bound receptor plus the un-bound receptor) (equation 1). It is related to the dissociation constant (K_{D}), which is defined as the product of the concentrations of the free ligand and the free receptor concentration divided by the concentration of the receptor-ligand complex (equation 2). After rearranging the equations, occupancy equals the concentration of the ligand divided by the quantity, K_{D} plus the concentration of the ligand (equation 3). K_{D} values for each trophic factor receptor were identified through the PubMed search engine. Only trophic factor receptors specific to the retina, RPE, and the choroid were included. Potential biological activity was only assumed from the calculated changes in trophic factor receptor occupancies and did not mathematically factor into the calculations.

Occupancy=[RL]/[RL+R], Equation 1
where R—unbound receptor, L—ligand, and RL—receptor-ligand complex

Dissociation constant(K_{D})=[R][L]/[RL], Equation 2
where R—receptor, L—ligand, and RL—receptor-ligand complex

Receptor occupancy=[L]/[K_{D}+L], Equation 3
where L—ligand and K_{D}—dissociation constant

Statistical Analysis

All analysis was performed using Sigma Plot 11 from Systat Software Inc., San Jose, Calif. Significance was accepted at p<0.05. If the data passed the Shapiro-Wilks normality test and the Equal Variance test, an un-paired t-test (two groups) or a one-way analysis of variance (ANOVA) followed by the Holm-Sidak all pairwise comparison method (multiple groups) was used. However, if the data failed either the normality or the variance test, then the non-parametric Mann-Whitney Rank Sum test (two groups) or the non-parametric Kruskal-Wallis one-way ANOVA on ranks followed by the Dunn's method for pairwise multiple comparisons (multiple groups) was used. Potential correlation of secretion of trophic factors to each other and to death-to-preservation and death-to-receipt times were calculated using the Spearman rank order correlation.

### Results

Trophic Factor Protein Secretion

The mean±SEM micrograms of RPE protein isolated from adult, AMD, and fetal eye-cups was 1.47±0.05, 1.38±0.05, and 0.14±0.03, respectively. Four trophic factors were detected in ≧85% of CM samples from adult, AMD, and fetal eye-cups—HGF, BDNF, EGF, and CNTF. The secretion of BDNF (expressed as the mean±SEM picograms per microgram of RPE protein) by AMD eye-cups (AMD-CM) was significantly higher than that from non-AMD eye-cups (adult-CM) (Table 5). The secretion of HGF and PEDF was significantly higher by fetal eye-cups (fetal-CM) as compared with adult eye-cups (adult-CM) (Table 5). For fetal-CM, neither gestational age nor gender consistently affected the trophic factor secretion of all tested factors. Neither death-to-receipt nor death-to-preservation time correlated significantly with trophic factor secretion from adult or AMD eye-cups. These correlations were not calculated for fetal eye-cups due to lack of information from the eye banks. significantly with trophic factor secretion from adult or AMD eye-cups. These correlations were not calculated for fetal eye-cups due to lack of information from the eye banks.

Kruskal-Wallis ANOVA on Ranks followed by Dunn's Method for Pairwise Comparison showed that the secretion of BDNF by AMD vs. fetal eye-cups was significantly different (p<0.001). The change in BDNF secretion represents a disease-specific alteration in trophic factor production since there were no significant differences in age between adult and AMD donor eyes (p=0.243 by Mann-Whitney Rank Sum test).

TABLE 5
Trophic factor quantification of adult, AMD, and fetal eye-cups.

| Trophic | Adult (n = 12) | | | AMD (n = 10) | | | Fetal (n = 23) | | |
|---|---|---|---|---|---|---|---|---|---|
| Factor | Ave | SEM | Range | Ave | SEM | Range | Ave | SEM | Range |
| HGF | 7950.0 | 2488.6 | 663.8-42134.5 | 7818.9 | 2360.7 | 829.8-30752.3 | 12440.7^{†} | 1734.1 | 2067.9-37207.8 |
| BDNF | 225.1 | 59.6 | 31.0-1516.4 | 514.1* | 112.3 | 2.5-1595.7 | 265.4 | 35.9 | 31.6-867.0 |
| EGF | 6.6 | 2.0 | 1.0-23.8 | 9.6 | 1.8 | 2.4-28.1 | 8.6 | 1.5 | 1.0-32.1 |
| PEDF | 1859678 | 692752 | 235122-9419625 | 893423.0* | 229892 | 166538-2577187 | 7382019^{‡} | 878926 | 459300-22612615 |

Mean, SEM, and range of concentrations of trophic factors (picograms) in adult-, AMD-, and fetal-CM quanified by multiplex ELISA.
AMD- and fetal-CM values were compared to adult-CM values for statistical significance (p < 0.05) using the non-parametric Mann-Whitney Rank Sum test.
*p = 0.027,
^{†}p = 0.018, and
^{‡}p < 0.001.

Trophic Factor mRNA Expression

To determine whether there was a correlation between trophic factor mRNA expression and protein secretion, RNA was isolated from normal and AMD adult, and fetal RPE cells. Real-time PCR (Table 7) showed that RPE cells from AMD donor eye-cups had significantly lower levels of transcripts for EGF than RPE from adult donor eye-cups. The levels of all four transcripts (HGF, BDNF, EGF, and PEDF) were significantly different in fetal vs. adult RPE cells isolated from donor eyes. In addition, the level of PEDF was significantly higher in AMD vs. fetal RPE cells. Transcripts were detected 100% of the time in fetal RPE cells while BDNF was only detected in seven of eight adult samples.

TABLE 6
Trophic factor mRNA expression of adult, AMD, and fetal RPE cells.

| | Mean ± SEM transcript levels | | | p-value | p-value | p-value |
|---|---|---|---|---|---|---|
| | (EGF_{adult} = 1) | | | (adult | (adult | (AMD |
| Trophic | Adult RPE | AMD RPE | Fetal RPE | vs. | vs. | vs. |
| Factor | (n = 8) | (n = 5) | (n = 16) | AMD) | fetal) | fetal) |
| HGF | 0.002 ± 0.0007 | 0.05 ± 0.05 | 0.04 ± 0.008 | NS | <0.001 | NS |
| BDNF | 0.0007 ± 0.0004* | 0.002 ± 0.001 | 0.002 ± 0.0003 | NS | <0.001 | NS |
| EGF | 1.0 ± 0.4 | 0.4 ± 0.2 | 0.01 ± 0.002 | 0.005 | <0.001 | NS |
| PEDF | 408.8 ± 73.6 | 370.8 ± 56.0 | 139.5 ± 6.3 | NS | <0.001 | <0.001 |

Abbreviations:
NS, not significant.
Values were compared for significance (p < 0.05) using an unpaired t-test.
*Represents values based on n = 7.

Contribution of Bruch's Membrane-Choroid-Sclera to Trophic Factor Secretion

To determine whether RPE cells (vs. other constituents of the Bruch's membrane explants such as choroid-sclera) were the main source of the detected trophic factors in adult- and fetal-CM, RPE cells were gently brushed off Bruch's membrane at time 0, and CM was collected from choroid-sclera eye-cups. Three factors (VEGF-A, HGF, and heparin binding-epidermal growth factor [HB-EGF]) identified in adult- and fetal-CM were also identified in BrM-C-S-CM. (VEGF-A and HB-EGF were not included in our study due to <85% identification in adult- or fetal-CM samples.) The relative amount (mean±SEM) of HGF in BrM-C-S-CM compared to adult- and fetal-CM was 0.47±0.18 and 0.02±0.005, respectively. Of note, the relative levels (mean±SEM) of VEGF-A in BrM-C-S-CM to those found in 95.8% of adult-CM and 78.1% fetal-CM samples were 3.04±0.66 and 2.07±0.29, respectively. Trophic factor secretion from BrM-C-S of AMD eyes was not studied since the trophic factors that varied significantly among adult- and AMD-CM (Table 5) were not produced by BrM-C-S of adult eyes. The relative (mean %) bestrophin levels found in adult and fetal BrM-C-S preparations to RPE cell isolations were 0.08% and 1.2%, respectively. These percentages may be due to non-specific binding of proteins to the Bruch's membrane after RPE cell removal or to a very small number of RPE cells that were not brushed off.

Preservation of Porcine Retina

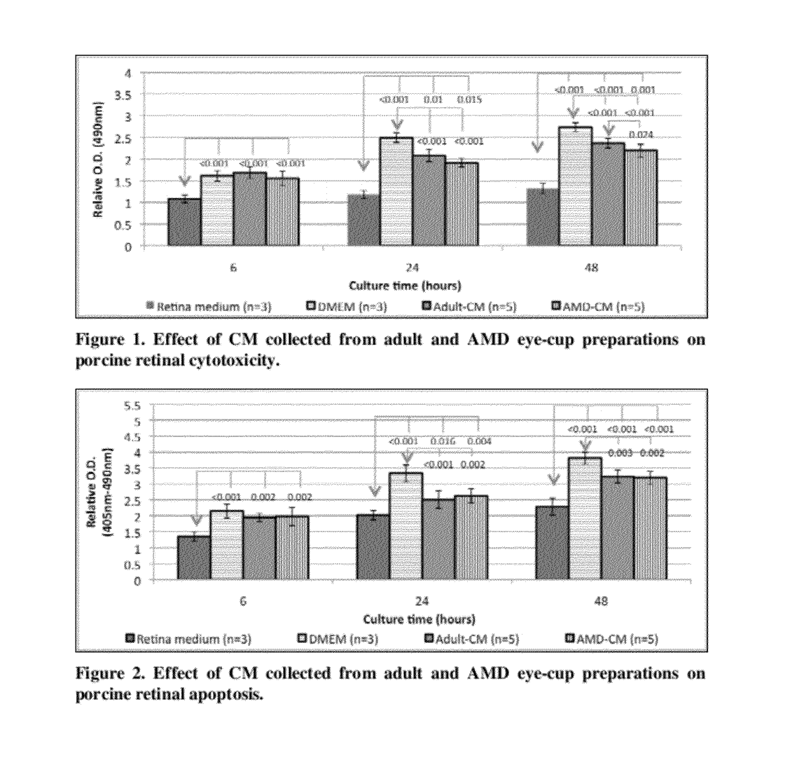

To determine if CM from the three different eye-cup preparations affect retinal preservation to different degrees, 6-millimeter retinal explants were isolated from porcine eyes and cultured from 1-48 hours in retina medium (positive control), DMEM (negative control), and eye-cup CM (adult-CM, AMD-CM, and fetal-CM). LDH and DNA fragmentation were measured at each time point with the 1-hour results serving as the reference time point. At each time point, retinae in adult- and AMD-CM showed significantly lower survival than the retinae in retina medium, significantly better survival than retinae in DMEM, and did not differ significantly from one another (FIGS. 1 and 2).

FIG. 1 illustrates the effect of CM collected from adult and AMD eye-cup preparations on porcine retinal cytotoxicity. Porcine retina was cultured in CM collected from eye-cups of adult (n=5; adult-CM) and AMD (n=5; AMD-CM) eyes, and in positive (n=3; retina medium) and negative (n=3; DMEM) controls. Mean±SEM LDH concentration was compared for statistical significance (p<0.05) using a one-way ANOVA followed by a Holm-Sidak method for all pairwise multiple comparison.

FIG. 2 illustrates the effect of CM collected from adult and AMD eye-cup preparations on porcine retinal apoptosis. Porcine retina was cultured in CM collected from eye-cups of adult (n=5; adult-CM) and AMD (n=5; AMD-CM) eyes, and in positive (n=3; retina medium) and negative (n=3; DMEM) controls. Mean±SEM DNA fragmentation was compared for statistical significance (p<0.05) using a one-way ANOVA followed by a Holm-Sidak method for all pairwise multiple comparison.

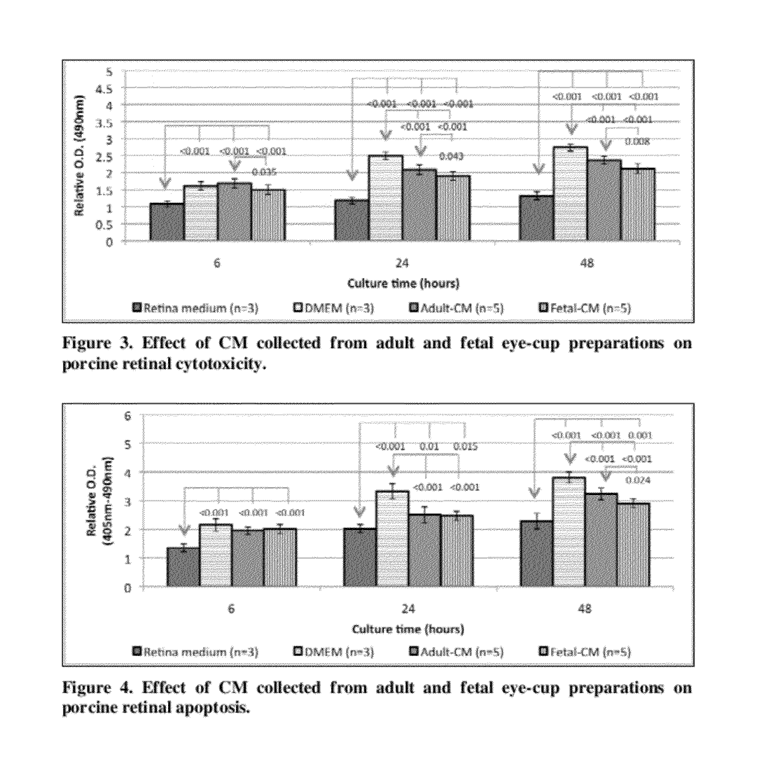

Except for the 6-hour time point, adult- and fetal-CM induced significantly better retinal survival than DMEM (FIGS. 3 and 4).

FIG. 3 illustrates the effect of CM collected from adult and fetal eye-cup preparations on porcine retinal cytotoxicity. Porcine retina was cultured in CM collected from eye-cups of adult (n=5; adult-CM) and fetal (n=5; fetal-CM) eyes, and in positive (n=3; retina medium) and negative (n=3; DMEM) controls. Mean±SEM LDH concentration was compared for statistical significance (p<0.05) using a one-way ANOVA followed by a Holm-Sidak method for all pairwise multiple comparison.

FIG. 4 illustrates the effect of CM collected from adult and fetal eye-cup preparations on porcine retinal apoptosis. Porcine retina was cultured in CM collected from eye-cups of adult (n=5; adult-CM) and fetal (n=5; fetal-CM) eyes, and in positive (n=3; retina medium) and negative (n=3; DMEM) controls. Mean±SEM DNA fragmentation was compared for statistical significance (p<0.05) using a one-way ANOVA followed by a Holm-Sidak method for all pairwise multiple comparison.

In addition, fetal-CM was significantly better than adult-CM at reducing retinal cytotoxicity (at all time points) and apoptosis (at the 48-hour time point only).

Trophic Factor Receptor Occupancy

Compared to CM isolated from adult eyes, concentrations of BDNF and EGF were significantly higher in AMD-CM and concentrations of HGF, BDNF, and PEDF were significantly higher in fetal-CM. (Table 7).

TABLE 7
Trophic factor concentrations and receptor occupancies.⁶⁰⁻⁶⁴

| | | Trophic Factor Concentration (pM), mean ± | | |
|---|---|---|---|---|
| Trophic | Receptor* | SEM (% Occupancy, mean ± SEM) | | |
| Factor | (K_{D} [pM]) | Adult (n = 12) | AMD (n = 10) | Fetal (n = 23) |
| HGF | c-Met (20-30)^{†} | 72.8 ± 22.8 | 74.5 ± 24.5 | 135.8 ± 18.9^{§} |
| | | (70.8 ± 5.3) | (71.3 ± 5.4) | (81.9 ± 6.7) |
| BDNF | TrkB (1000) | 11.8 ± 3.1 | 32.3 ± 7.1^{§} | 15.6 ± 2.1^{‡} |
| | | (1.2 ± 0.2) | (3.1 ± 0.7) | (1.5 ± 0.2) |
| | p75^{NTR} (1300) | 11.8 ± 3.1 | 32.3 ± 7.1^{§} | 15.6 ± 2.1^{‡} |
| | | (0.9 ± 0.2) | (2.4 ± 0.5) | (1.2 ± 0.1) |
| EGF | EGF-R (700) | 0.8 ± 0.1 | 1.4 ± 0.3^{‡} | 1.2 ± 0.2 |
| | | (0.1 ± 0.02) | (0.2 ± 0.04) | (0.2 ± 0.02) |
| PEDF | PEDF-R (2500-6500)^{†} | 27491 ± 10240 | 17261 ± 4442 | 125671 ± 14963^{§} |
| | | (80.9 ± 4.4) | (72.6 ± 4.3) | (95.1 ± 0.5) |

*Includes receptors specific to retina, RPE, and choroid only.
^{†}Calculations based on K_{D} values of 30 pM (c-Met) and 6500 pM (PEDF-R), respectively. Significantly different (t-test) from adult eyes at
^{‡}p < 0.05,
^{§}p < 0.001.

### Example 2

### Methods and Materials

Human Donor Eye Tissue

Eyes from non-AMD and AMD Caucasian donors of 55 years of age or older were obtained through various eye banks or suppliers (National Disease Resource Interchange, Inc., Philadelphia, Pa.; Vision Share, Apex, N.C.; Midwest Eye banks, Ann Arbor, Mich.; Tampa Lions Eye Institute for Transplant & Research, Tampa, Fla.; San Diego Eye bank, San Diego, Calif.) in the United States and Canada. The tissue acceptance criteria included: 1) no recent history of chemotherapy or radiation to the head; 2) not on a ventilator prior to death; 3) up to seven hours from death to enucleation with eyes preserved in a moist chamber and stored on ice immediately after removal; 4) no more than 48 hours from death to receipt; and 5) intact, bright, not opaque, orange-colored RPE monolayer as visualized through a dissecting microscope. Fetal eyes (17-22 weeks gestation) were obtained through Advanced Bioscience Resources, Inc (ABR; Alameda, Calif.). By these gestational ages, the RPE cell monolayer is formed completely. Upon arrival, eyes were cleaned of extraneous tissue, dipped in betadine solution (10% Povidone-iodine, The Purdue Frederique Company, Stamford, Conn.) which was immediately rinsed off with balanced salt solution, and washed twice for 10 minutes at 4° C. in Dulbecco's Modified Eagle Medium (DMEM with one g/l glucose, L-glutamine and sodium pyruvate, containing 3.7 g/L sodium bicarbonate) (Cellgro-Mediatech Inc., Manasses, Va.) supplemented with 250 μg/ml amphotericin B (Gibco-Invitrogen, Carlsbad, Calif.).

RPE Cell Isolation

Anterior segment and neural retina were removed, exposing the posterior segment. RPE-choroid was separated from the sclera and incubated in 0.8 mg/ml collagenase IV (Sigma-Aldrich, St. Louis, Mo.) for 60 minutes at 37° C., 10% CO₂ (fetal posterior segments) or 0.4 mg/ml collagenase IV for 30 minutes at 37° C., 10% CO₂ (adult posterior segments). RPE sheets were separated carefully from the choroid with a 22-gauge needle, rinsed in Dulbecco's Phosphate Buffered Saline (DPBS; Cellgro-Mediatech Inc.), cut up into small pieces (fetal RPE) or triturated with a 200 μl pipette (adult non-macular RPE), and plated on 35 mm tissue culture treated (by vacuum glass plasma) culture dishes (TCTP), coated with bovine corneal endothelium-extra cellular matrix (BCE-ECM) (adult and fetal RPE) or uncoated TCTP dishes (fetal RPE only) (FALCON; Becton Dickinson Labware Company, Franklin Lakes, N.J.). Homogenous RPE cell population was verified by morphology and cytokeratin staining according to manufacturers instructions (Sigma-Aldrich). BCE-ECM-coated TCTP dishes were prepared according to previously established methods. Cells were cultured in DMEM supplemented with 15% fetal bovine serum (FBS; Gibco-Invitrogen), one ng/ml of human recombinant bFGF (Gibco-Invitrogen), two mM L-glutamine (Gibco-Invitrogen), 2.50 μg/ml amphotericin B (Gibco-Invitrogen), and 0.05 mg/ml gentamicin sulfate (Cellgro-Mediatech Inc.). This medium, hereafter known as ‘RPE medium’, was changed three times a week. Upon reaching confluence (12-16 days after plating), fetal RPE cells were detached from dishes with 0.25% Trypsin-EDTA treatment for ˜7 minutes at 37° C., 10% CO₂. Viable cells were counted (Trypan Blue Solution, Cellgro-Mediatech Inc.) under a light microscope (Standard 20; Carl Zeiss, Oberkochen, Germany), and 5×10⁵ cells were seeded on either BCE-ECM-coated or uncoated TCTP dishes (passage-1 fetal RPE cells). Upon confluence and prior to subsequent passaging, passage-1 fetal RPE cells were cultured in DMEM for 24 hours in order to synchronize the cell cycle.²⁴ Cultured passage-2 to passage-6 fetal RPE cells were used for all experiments. Adult RPE cells were not passaged (‘primary’ culture).

Conditioned Medium (CM) Collection

Primary adult RPE cells (n=7; average age, 81.7 years) were isolated from donor eyes without subretinal pathology (based on biomicroscopy with a dissecting microscope) and cultured in 35 mm dishes for 12-16 days (until visually-determined cessation of cell division). Passage-2, -4, and -6 fetal RPE cells (n=5; average age, 19.0 weeks gestation) were seeded at 1.2×10⁶ cells per well (½ of in situ RPE density) in 12-well BCE-ECM-coated or uncoated TCTP plates (Costar; Corning Inc., Corning, N.Y.) and allowed to grow for 6 or 29 days. Confluence was reached after 3-4 days. Cells were photographed prior to media collection. RPE medium was changed three times a week. Quiescent adult RPE and passage-2, -4, and -6 fetal RPE cells after 6 and 29 days in culture were washed twice with DPBS to remove any remnants of FBS and bFGF found in RPE medium and cultured in 0.21 ml/cm² (adult RPE) or 0.39 ml/cm² (fetal RPE) of DMEM for 24 hours at 37° C., 10% CO₂. The resultant CM (cultured adult RPE-CM; and passage-2, day-7; passage-2, day-30; passage-4, day-7; passage-4, day-30; passage-6, day-7; and passage-6 day-30 cultured fetal RPE-CM, respectively) was collected, centrifuged at 1000 rpm for five minutes (Model 5415c, Eppendorf, Hauppauge, N.Y.) to remove cellular debris, and frozen at −80° C. (Bio Freezer, Form a Scientific, Evanston, Ill.).

Trophic Factor Quantification

Preliminary studies testing secretion of trophic factors (previously shown to possess photoreceptor and/or retina-preserving functions) determined that nine trophic factors (Table 8) were consistently secreted by fetal RPE cells at a concentration >10 pg/μg RPE protein (epidermal growth factor [EGF], glial-derived neurotrophic factor [GDNF], PDGF-β, NT-3, and interleukin 1-beta [IL-1β] were excluded from quantification). Preparations of CM were analyzed for these nine factors in duplicate via multiplex ELISA (Aushon Biosystems, Woburn, Mass.). DMEM was analyzed for the same factors as a control for non-specific binding (quantified in duplicate on three separate occasions to ensure method reliability). CM from cultured RPE cells were corrected for these values. Mean±SEM for each trophic factor above threshold detection level was expressed as picograms of factor per microgram of RPE protein. Trophic factor detection frequencies, defined as the number of times a trophic factor was successfully identified by multiplex ELISA divided by the total number of CM samples analyzed, were calculated.

TABLE 8
Retinal and photoreceptor-preserving trophic
factors quantified by multiplex ELISA.²⁶⁻³¹

| | MW | |
|---|---|---|
| Trophic factor | (kDa) | Biological Effect* |
| Brain-derived neurotrophic factor | 14 | Neurotrophic |
| (BDNF) | | |
| Ciliary neurotrophic factor (CNTF) | 24 | Neurotrophic |
| Basic fibroblast growth factor (bFGF) | 18 | Pro-angiogenic, |
| | | photoreceptor rescue |
| Heparin-binding epidermal growth | 23 | RPE proliferation, |
| factor (HB-EGF) | | VEGF secretion |
| Hepatocyte growth factor (HGF) | 83 | RPE survival, |
| | | neuroprotective |
| Nerve growth factor (NGF) | 13 | Neurotrophic, inflammation |
| Pigment epithelium-derived factor | 46 | Anti-angiogenic, |
| (PEDF) | | neurotrophic |
| Vascular endothelial growth factor-A | 43 | Pro-angiogenic, |
| (VEGF-A) | | photoreceptor development |
| Leukemia inhibitory factor (LIF) | 20 | Photoreceptor rescue, |
| | | RPE survival |

Abbreviations: MW, molecular weight.
*Only trophic effects are listed.

Protein Quantification

Protein was isolated from RPE cells after the 24-hour CM collection period. After the 35 mm culture dishes (cultured adult RPE cells) and 12-well plates (cultured fetal RPE cells) were washed twice with ice-cold DPBS, 200 μl of 1× lysis buffer (10 mM Tris, 500 μM EDTA, 75 mM NaCl, 0.5% Triton X-100, 5% glycerol, and 1% 100× protease inhibitor cocktail (Pierce-Thermo Fischer Scientific, Rockford, Ill.) prepared in dH₂O) was added. RPE cells were gently brushed off from the choroid into the lysis buffer, triturated on ice, sonicated three times for 10 seconds at 4° C. (Branson Sonifier 250; VWR Scientific, West Chester, Pa.), and centrifuged for 10 minutes at 10000 rpm, 4° C. The lysates were collected and frozen at −80° C. Protein was quantified using the Bradford reagent (Sigma-Aldrich, St. Louis, Mo.) according to manufacturer's instructions.

### Porcine Retina

Porcine Eye Tissue:

Eyes from 5- to 9-month, 150-230 lb, male and female American Yorkshire pigs were obtained from a local abattoir within three hours of enucleation (transported on ice). Porcine eyes were prepared for dissection by the method outlined previously for human donor eyes. Anterior segment and vitreous were removed, leaving the neural retina in the eye-cup. Six millimeter trephine blades (Storz Ophthalmic-Bausch and Lomb, Manchester, Mo.) were used to isolate equatorial, full-thickness retina tissue explants (avoiding the peripapillary region) by separating the retina from the underlying RPE-choroid-sclera. These explants were randomly assigned to different culture conditions in order to negate the potential effects of selection bias and variability in retinal thickness.

Retinal Cytotoxicity:

The lactate dehydrogenase (LDH) in vitro toxicology assay (TOX-7; Sigma-Aldrich) was used to assess the effects of various CM on retinal membrane integrity. Media from retinal explants collected at 1, 6, 24, and 48 hours of culture was centrifuged for five minutes at 1000 rpm to remove cellular debris. The supernatant was frozen at −20° C. and processed as per manufacturer's instructions. Colorimetric absorbances were assessed by a microplate reader (ELx800, BioTek, Winooski, Vt.) at 490 nm. The data are expressed relative to the 1-hour levels.

Retinal Apoptosis:

A cell death detection ELISA assay kit (Roche Diagnostics, Piscataway, N.J.) was used to quantify the effects of various CM on the amount of retinal DNA fragmentation. After 1, 6, 24, or 48 hours of culture, the explants were homogenized in 200 μl of provided lysis buffer by trituration, allowed to react for 30 minutes at room temperature, centrifuged for five minutes at 1000 rpm, 4° C., and the supernatant was frozen at −20° C. The specimens were processed as per manufacturer's instructions. Absorbances were measured at 405 nm with reference wavelength at 490 nm (ELx800, BioTek). A DNA-histone complex (included) served as the positive control. The data are expressed relative to the 1-hour levels.

Real-Time Polymerase Chain Reaction (Real-Time PCR):

RNA was isolated from cultured porcine retina and quantified for trophic factor mRNA expression. Briefly, RNA was washed, bound, and eluted according to manufacturer's instructions (RNeasy Mini Kit, Qiagen). It was quantified using a spectrophotometer (Nanodrop-1000, Thermo Fisher Scientific, Waltham, Mass.). cDNA was prepared using an RT-PCR reaction mixture (Applied Biosystems, Foster City, Calif.) in a thermocycler (MJ Mini Personal Thermo Cycler, Bio-Rad, Hercules, Calif.). Real-time PCR for each trophic factor was performed on real time PCR System (Model 7500, Applied Bioscience). 18S rRNA transcript served as an endogenous control. Trophic factors were expressed in relation to the arbitrarily chosen heparin binging-epidermal growth factor (HB-EGF)ₜᵢₘₑ₌₀ transcript, which was assigned a value of one. The results were calculated for each group of samples as mean±SEM of the level of HB-EGFₜᵢₘₑ₌₀.

Fetal RPE—Porcine Retina Co-Culture

Passage-2 and passage-6 fetal RPE cells were isolated as described previously and seeded in 96-well TCTP plates (FALCON; Becton Dickinson Labware) at 3.26×10⁴ cells per well. RPE medium was changed three times a week. On day-7, cells were washed twice with DPBS, and 100 μl of DMEM, and one porcine retinal explant (six mm diameter) was added to each well. Control wells included cultured fetal RPE cells with no retina in DMEM or porcine retina in DMEM (no fetal RPE). Retinal cytotoxicity and apoptosis were measured at 1, 6, 24, and 48 hours. Cytotoxicity was corrected by subtracting the fetal RPE (n=5) contribution (control wells) from the total measurements. The 1-hour time point served as an internal control of retinal preservation. CM was collected 24 and 48 hours after culture from wells containing fetal RPE cells and porcine retina (fetal RPE-retina-CM), wells with fetal RPE cells only (fetal RPE-CM), and wells with porcine retina only (retina-CM). CM was centrifuged, frozen, and analyzed for selected trophic factors (Table 8). To determine if alterations of porcine retinal trophic factor mRNA expression correlated with changes in protein secretion (i.e., retinal trophic factor contribution to fetal RPE-retina-CM), retinae were harvested after co-culture with the fetal RPE cells at time 0 and 1, 6, 24, and 48 hours for mRNA quantification. 18S rRNA transcript served as an endogenous control with the VEGF-A transcript arbitrarily set to equal one (ΔCt method).

Isobaric Tag for Relative and Absolute Quantification (iTRAQ)

iTRAQ multiplex global protein analysis allows for simultaneous identification and quantification of proteins from different sources in one experiment. iTRAQ analysis was performed to determine if factors, in addition to those tested, that possess the potential to affect photoreceptor and retinal preservation could be identified in the CM. Four different preparations of primary cultured adult RPE-CM and passage-2, day-7 cultured fetal RPE-CM (ten ml of each medium) were processed. Detailed methods for iTRAQ analysis have been described in Liu T, et al. J Proteome Res 2007; 6:2565-2575. Briefly, after acetone precipitation of the proteins, the samples were desalted using a 2D gel Clean up Kit (Bio-Rad). The protein pellets were resuspended in 60 μl of a lysis buffer containing 150 mM triethylammonium bicarbonate (TEAB), 1% NP40 (Igepal CA-630; Sigma-Aldrich), 1% Triton X-100, and three μl of phosphatase inhibitor cocktail I and II. Protein concentrations were measured using the bicinchoninic acid (BCA) method (BCA Protein Assay Kit; Thermo Fisher Scientific) according to the manufacturer's instructions. The iTRAQ labeling procedures (using amine-specific, stable isotope reagents) were performed according to the manufacturer's instructions (Applied Biosystems) using equal volumes of each medium. Protein enzymatic digestion was performed by addition of eight μg of trypsin (Promega Corporation, Madison, Wis.) to each of the eight samples at 37° C., overnight. Peptides derived from four cultured adult RPE-CM were labeled with iTRAQ tags 113, 114, 115, and 116 whereas samples obtained from four cultured fetal RPE-CM were labeled with tags 117, 118, 119, and 121. The labeled peptides were mixed, loaded onto a strong cation exchange column (BioCAD Perfusion Chromatography System; Applied Bioscience), and detergents and free iTRAQ reagents were washed out. Labeled peptides were fractionated and analyzed on a tandem mass spectrometer (4800 Proteomics Analyzer MALDI-TOF-TOF, ABI). To reduce the probability of false identification, only proteins with at least two peptides with confidence values ≧95% were reported. Relative quantification of peptides in each sample was calculated from the areas under the peaks. Average fold changes between the adult and fetal RPE-CM were calculated. Scaffold Q+ software (Proteome Software Inc., Portland, Oreg.) was used to visualize peptide change across samples and sort differentially-expressed proteins. Ingenuity Pathway analysis software version 8.7 (Ingenuity Systems, Inc., Redwood City, Calif.) was used to identify the functional location (i.e., intracellular vs. secreted) of identified proteins.

Trophic Factor Receptor Occupancy

In order to relate the differences in the measured concentrations of secreted trophic factors in various CM to potential biological activity (defined as the ability of the CM to improve the survival of degenerating porcine retina), we calculated mean (±SEM) percent receptor occupancy for each trophic factor and its primary receptor. Passage-6, day-7 cultured fetal RPE-CM and cultured adult RPE-CM trophic factor receptor occupancies were compared to passage-2, day-7 cultured fetal RPE-CM values in a pairwise manner in order to elucidate statistically significant differences. Briefly, in occupancy theory, the magnitude of a biological response is posited to be directly proportional to the receptor-ligand complex concentration. It is also a function of the dissociation constant (K_{D}). Occupancy equals the concentration of the ligand divided by the quantity K_{D} plus the concentration of the ligand (equation 1). K_{D} values for each trophic factor receptor were identified through the PubMed search engine.

Receptor occupancy=[L]/[K_{D}+L], where L—ligand concentration and K_{D}—dissociation constant (equation 1)

Statistical Analysis

Trophic factors found to be below the factor-specific detection threshold were not included in statistical analyses. All CM analysis (except for the iTRAQ data) was performed using Sigma Plot 11 from Systat Software Inc., San Jose, Calif. Significance was accepted at p<0.05. After the data passed the Shapiro-Wilks normality test and the Equal Variance test, an un-paired t-test (two groups) or a one-way analysis of variance (ANOVA) followed by the Holm-Sidak all pairwise comparison method (multiple groups) was used. Potential correlation of secretion of trophic factors to each other and to death-to-preservation and death-to-receipt times were calculated using the Spearman rank order correlation. Comparison of adult vs. fetal iTRAQ data was performed using the 2-tailed t-test for each peptide (Excel; Microsoft Corporation, Redmond, Wash.).

### Results

Trophic Factor Protein Secretion

The mean±SEM micrograms of RPE protein isolated from fetal RPE cells of passage-2, -4, and -6 on day-7 and -30 grown on uncoated (TCTP) and BCE-ECM-coated dishes was 0.23±0.027 and 0.25±0.023, respectively. Fetal RPE cell culture purity was confirmed by cytokeratin staining of passage-2, day-7 cells grown in 12-well TCTP plates. Trophic factor secretion for different passages and times in culture is shown for fetal RPE grown on uncoated (Table 9) and BCE-ECM-coated (Table 10) TCTP dishes. For cells grown on TCTP dishes (Table 9), passage number and time in culture affected the secretion of some trophic factors including: 1) bFGF (passage-4, day-7 and passage-6, day-7 were significantly higher than other passages); 2) VEGF-A (passage-2, day-7 was significantly higher than passage-6, day-7); 3) PEDF (passage-2, day-30 was significantly higher than passage-4, day-7 and passage-6, day-7). For cells grown on BCE-ECM-coated dishes (Table 10), passage number and time in culture affected the secretion of the following trophic factors: 1) bFGF (passage-4, day-7 and passage-6, day-7 were significantly higher than other passages) and 2) CNTF (passage-4, day-7 and passage-6, day-7 were significantly higher than passage-2, day-7, passage-4, day-30, and passage-6, day-30). A paired t-test was used to compare fetal RPE trophic factor secretion within each passage and duration of culture as a function of the underlying substrate. The secretion of the following factors was significantly higher for fetal RPE on TCTP vs. BCE-ECM-coated dishes: 1) hepatocyte growth factor (HGF) (p=0.045) for passage-2, day-7 cells and 2) CNTF (p=0.009) for passage-6, day-30 cells. The secretion of the following factors was significantly higher for fetal RPE cells on BCE-ECM-coated vs. TCTP dishes: 1) HB-EGF (p=0.017) for passage-2, day-7 cells; 2) HB-EGF (p=0.032) for passage-2, day-30 cells; 3) PEDF (p=0.05) for passage-4, day-7 cells; 4) HB-EGF (p=0.024) for passage-4, day-30 cells; and 5) nerve growth factor (NGF) (p=0.003) for passage-4, day-30 cells. The trophic factor detection frequencies for cells grown on TCTP and BCE-ECM-coated dishes are listed in Table 11.

The Spearman rank order correlation was used to identify potential correlations in trophic factor secretion. The following high-degree (ρ>0.7) correlations were identified: 1) bFGF and VEGF-A in CM isolated from passage-2, day-7 fetal RPE cells grown on TCTP dishes (inverse correlation); 2) bFGF and HB-EGF in CM isolated from passage-6, day-30 fetal RPE cells grown on TCTP dishes; and 3) LIF and HB-EGF in CM isolated from passage-2, day-30 fetal RPE cells grown on BCE-ECM-coated dishes. The direct correlation between LIF and HB-EGF on photoreceptor/retinal preservation has not been established.

TABLE 9
Trophic factor composition of conditioned media collected from cultured fetal
RPE cells of different passages (passage-2, -4, or -6) and times in culture (Day-7 or -30)
grown on tissue culture-treated plastic dishes.

| Fetal RPE cell | | | | HB- | | |
|---|---|---|---|---|---|---|
| culture | | LIF | bFGF | EGF | HGF | VEGF-A |
| P2D7 | Mean ± | 9 ± 4.1 | 86.8 ± 14.9*^{,†} | 78.3 ± 17.1 | 807.8 ± 92.6 | 21518.8 ± 2666.2^{‡‡} |
| | SEM | | | | | |
| | Range | 4.6-17.2 | 55.5-137.7 | 33-122 | 628.7-938.1 | 15390-27596.9 |
| P2D30 | Mean ± | 6.6 ± 2.2 | 65.4 ± 6.2 | 95.6 ± 17.3 | 557 ± 66.9 | 16673.7 ± 3456.2 |
| | SEM | | | | | |
| | Range | 2.4-9.6 | 48.6-79.4 | 42.3-137.3 | 489.5-690.8 | 9780-24296.8 |
| P4D7 | Mean ± | 8.9 ± 2.6 | 171.4 ± 19.2^{‡,§,\|\|} | 75.3 ± 8.5 | 500 ± 82.2 | 10045.7 ± 1664.5 |
| | SEM | | | | | |
| | Range | 2.8-14.4 | 132.8-238.7 | 45.6-98.8 | 405.3-663.8 | 4829.8-14850 |
| P4D30 | Mean ± | 7 ± 1.5 | 57.3 ± 10 | 102.2 ± 16.6 | 725.5 ± 87.2 | 13732.2 ± 1892.5 |
| | SEM | | | | | |
| | Range | 2.9-9.8 | 37.2-94.3 | 45.9-131.2 | 638.2-812.7 | 8423.9-18652.5 |
| P6D7 | Mean ± | 10.7 ± 2.9 | 160.4 ± 20.4^{#,}**^{,††} | 81.3 ± 12.9 | 617.8 ± 44.9 | 8563.6 ± 1368.6 |
| | SEM | | | | | |
| | Range | 4.1-18.4 | 110.9-200.7 | 46.8-106.8 | 533.5-712.1 | 5334-12171 |
| P6D30 | Mean ± | 8.5 ± 3.4 | 70.3 ± 10.4 | 102.2 ± 13.4 | 771.5 ± 71.4 | 11691.7 ± 2221.4 |
| | SEM | | | | | |
| | Range | 2.3-17.1 | 38.7-102.8 | 62.7-138.9 | 629.4-855.1 | 4014.5-17439.2 |

| Fetal RPE cell | | | | | |
|---|---|---|---|---|---|
| culture | | NGF | BDNF | CNTF | PEDF |
| P2D7 | Mean ± | 13 ± 2.6 | 246.8 ± 35.5 | 34.5 ± 6.7 | 845334 ± 89237 |
| | SEM | | | | |
| | Range | 9.9-18.3 | 146.1-298.8 | 27.8-41.1 | 627720-992325 |
| P2D30 | Mean ± | 15.1 ± 2.1 | 263.5 ± 31.3 | 37.2 ± 6.5 | 1045309 ± 119245^{§§,\|\|} |
| | SEM | | | | |
| | Range | 11-22.7 | 184.1-356.9 | 29-50 | 820748-1373697 |
| P4D7 | Mean ± | 11.5 ± 2.3 | 171.3 ± 36.8 | 58.2 ± 11.3 | 483253 ± 59223 |
| | SEM | | | | |
| | Range | 7.5-17.5 | 84.9-263.1 | 35.7-70.3 | 469635-608933 |
| P4D30 | Mean ± | 9.2 ± 1.8 | 248.2 ± 45.8 | 50.7 ± 10.4 | 721518 ± 87148 |
| | SEM | | | | |
| | Range | 4.5-12.9 | 133.2-348 | 32-67.8 | 528855-887213 |
| P6D7 | Mean ± | 12.2 ± 2.5 | 138.8 ± 31.1 | 50.4 ± 3.1 | 483256 ± 63146 |
| | SEM | | | | |
| | Range | 6-17.4 | 49.5-182.4 | 44.1-56.3 | 383383-666180 |
| P6D30 | Mean ± | 17.6 ± 2.1 | 210.7 ± 38.2 | 54.4 ± 5.2 | 725030 ± 124978 |
| | SEM | | | | |
| | Range | 13.5-19.7 | 127.2-312 | 45.1-63 | 522720-1086026 |

Differences in trophic factor production with respect to passage number and duration of culture were evaluated for statistical significance (p < 0.05) using a one-way ANOVA followed by a Holm-Sidak method for all pairwise multiple comparisons.
The following comparisons were statistically significant:
*passage-2 day-7 vs. passage-4 day-7 (p < 0.001),
^{†}passage-2 day-7 vs. passage-6 day-7 (p = 0.002),
^{‡,§,||}passage-4 day-7 vs. passage-2 day-30, passage-4 day-30, and passage-6 day-30 (p < 0.001 for all),
^{#,}**^{,††}passage-6 day-7 vs. passage-2 day-30, passage-4 day-30, and passage-6 day-30 (p < 0.001 for all),
^{‡‡}passage-2 day-7 vs. passage-6 day-7 (p < 0.05),
^{§§}passage-2 day-30 vs. passage-4 day-7 (p < 0.001), and
^{||}passage-2 day-30 vs. passage-6 day-7 (p < 0.001).
Abbreviations:
P2D7, passage-2, day-7;
P2D30, passage-2, day-30;
P4D7, passage-4, day-7;
P4D30, passage-4, day-30;
P6D7, passage-6, day-7;
P6D30, passage-6, day-30;
LIF, leukemia inhibitory factor,
bFGF, basic fibroblast growth factor;
HB-EGF, heparin binding-epidermal growth factor;
HGF, hepatocyte growth factor;
VEGF-A, vascular endothelial growth factor-A;
NGF, neuronal growth factor;
BDNF, brain-derived neurotrophic factor;
CNTF, ciliary neurotrophic factor;
PEDF, pigment epithelium-derived factor.

TABLE 10
Trophic factor composition of conditioned media collected from cultured fetal
RPE cells of different passages (Passage-2, -4, or -6) and times in culture (Day-7 or -30)
grown on bovine corneal endothelial cell-extracellular matrix-coated dishes.

| Fetal RPE cell | | | | HB- | | |
|---|---|---|---|---|---|---|
| culture | | LIF | bFGF | EGF | HGF | VEGF-A |
| P2d7 | Mean ± | 8.2 ± 2.9 | 92.8 ± 18.9 | 149 ± 16.3 | 521.4 ± 63.2 | 19719.5 ± 1540.8 |
| | SEM | | | | | |
| | Range | 1.6-16.9 | 38.6-119.9 | 110.6-193.8 | 340.2-610.9 | 15309.1-24367.5 |
| P2D30 | Mean ± | 9.6 ± 1.8 | 60.4 ± 9.9 | 176.7 ± 26.1 | 760.7 ± 126.3 | 19390 ± 3651.1 |
| | SEM | | | | | |
| | Range | 3.3-13.4 | 31.7-93.5 | 113.5-270.6 | 412.8-1015 | 8452.5-24832.8 |
| P4D7 | Mean ± | 6.5 ± 2.1 | 177.6 ± 25*^{,†,‡,§} | 136.8 ± 25.5 | 488.3 ± 95.4 | 13256.4 ± 3010.5 |
| | SEM | | | | | |
| | Range | 3.2-12.6 | 94.2-248.1 | 75-196 | 242.2-617.4 | 5101.5-20475 |
| P4D30 | Mean ± | 9.7 ± 2.8 | 67 ± 16.8 | 177.2 ± 21.5 | 628.6 ± 83.2 | 18346.4 ± 2440.9 |
| | SEM | | | | | |
| | Range | 3.6-17.2 | 16.5-119.1 | 135-252.6 | 411.7-909.9 | 12318.3-26985 |
| P6D7 | Mean ± | 12.3 ± 2.9 | 216.8 ± 25^{\|\|,#,}**^{,††} | 118.7 ± 18.5 | 448.5 ± 92 | 9032 ± 1446.6 |
| | SEM | | | | | |
| | Range | 8.8-20.9 | 168.9-312 | 90-184.5 | 225.4-775.2 | 6029.4-13822.5 |
| P6D30 | Mean ± | 10.2 ± 2.3 | 82.7 ± 26.1 | 157.4 ± 29.6 | 545.4 ± 65.1 | 17715.7 ± 3817 |
| | SEM | | | | | |
| | Range | 3.6-14.2 | 40.4-185.5 | 86.1-240.4 | 355.4-698.2 | 7926.6-24982.5 |

| Fetal RPE cell | | | | | |
|---|---|---|---|---|---|
| culture | | NGF | BDNF | CNTF | PEDF |
| P2d7 | Mean ± | 14.6 ± 3.4 | 321.9 ± 37 | 35 ± 5.4 | 803526 ± 121280 |
| | SEM | | | | |
| | Range | 4.4-24.5 | 258.5-386.7 | 24.7-42.9 | 526845-1101413 |
| P2D30 | Mean ± | 22.3 ± 3.1 | 185.9 ± 31.5 | 41.9 ± 6.7 | 1033486 ± 190309 |
| | SEM | | | | |
| | Range | 14-27 | 154.4-217.4 | 28.9-60.5 | 657218-1544275 |
| P4D7 | Mean ± | 22.7 ± 5 | 282 ± 52.8 | 56.2 ± 5.5^{‡‡,§§,\|\|} | 671511.6 ± 49339 |
| | SEM | | | | |
| | Range | 13.7-32.4 | 229.2-334.8 | 42-74.7 | 579244-809888 |
| P4D30 | Mean ± | 28.2 ± 3.4 | 244.6 ± 28 | 33.8 ± 5.2 | 935137 ± 94989 |
| | SEM | | | | |
| | Range | 20-35.1 | 200.7-296.8 | 28.6-39 | 695988-1095681 |
| P6D7 | Mean ± | 25.6 ± 5.5 | 206.5 ± 30.8 | 58.2 ± 3.6^{##,}***^{,†††} | 440924 ± 64603 |
| | SEM | | | | |
| | Range | 14.9-38.2 | 175.7-237.4 | 54.6-61.8 | 323910-632376 |
| P6D30 | Mean ± | 32.6 ± 6 | 218.5 ± 32.5 | 26 ± 4.4 | 765632 ± 178870 |
| | SEM | | | | |
| | Range | 19.9-48.8 | 186-251 | 21.1-39.2 | 264750-1334755 |

Differences in trophic factor production with respect to passage number and duration of culture were evaluated for statistical significance (p < 0.05) using a one-way ANOVA followed by a Holm-Sidak method for all pairwise multiple comparisons. The following comparisons were statistically significant:
*^{,†,‡,§}passage-4 day-7 vs. passage-2 day-7 (p = 0.009), passage-2 day-30 (p < 0.001), passage-4 day-30 (p = 0.001), and passage-6 day-30 (p = 0.004),
^{||,#,}**^{,††}passage-6 day-7 vs. passage-2 day-7 (p < 0.001), passage-2 day-30 (p < 0.001), passage-4 day-30 (p < 0.001), and passage-6 day-30 (p < 0.001),
^{‡‡,§§,||}passage-4 day-7 vs. passage-2 day-7 (p = 0.018), passage-4 day-30 (p = 0.027), and passage-6 day-30 (p < 0.001), and
^{##,}***^{,‡‡‡} passage-6 day-7 vs. passage-2 day-7 (p = 0.034), passage-4 day-30 (p = 0.041), and passage-6 day-30 (p = 0.004).
Abbreviations:
P2D7, passage-2, day-7;
P2D30, passage-2, day-30;
P4D7, passage-4, day-7;
P4D30, passage-4, day-30;
P6D7, passage-6, day-7;
P6D30, passage-6, day-30;
LIF, leukemia inhibitory factor,
bFGF, basic fibroblast growth factor;
HB-EGF, heparin binding-epidermal growth factor;
HGF, hepatocyte growth factor;
VEGF-A, vascular endothelial growth factor-A;
NGF, neuronal growth factor;
BDNF, brain-derived neurotrophic factor;
CNTF, ciliary neurotrophic factor;
PEDF, pigment epithelium-derived factor.

TABLE 11
Detection frequencies of trophic factors identified in conditioned
media collected from cultured adult and fetal cells.

Detection frequency (%)

| | | |
|---|---|---|
| Trophic | Fetal RPE | Adult RPE |

| Factor | TCTP | BCE-ECM | BCE-ECM |
|---|---|---|---|
| LIF | 73 | 87 | 100 |
| bFGF | 97 | 100 | 100 |
| HB-EGF | 100 | 100 | 86 |
| HGF | 57 | 87 | 86 |
| VEGF-A | 100 | 100 | 100 |
| NGF | 77 | 83 | 100 |
| BDNF | 83 | 47 | 71 |
| CNTF | 57 | 67 | 57 |
| PEDF | 80 | 87 | 100 |

Abbreviations: TCTP, tissue culture treated plastic; BCE-ECM, bovine corneal endothelial cell-extracellular matrix; LIF, leukemia inhibitory factor, bFGF, basic fibroblast growth factor; HB-EGF, heparin binding-epidermal growth factor; HGF, hepatocyte growth factor; VEGF-A, vascular endothelial growth factor-A; NGF, neuronal growth factor; BDNF, brain-derived neurotrophic factor; CNTF, ciliary neurotrophic factor; PEDF, pigment epithelium-derived factor.

Secretion by cultured primary adult RPE cells was significantly higher for leukemia inhibitory factor (LIF), basic fibroblast growth factor (bFGF), and NGF and significantly lower for VEGF-A, BDNF, and PEDF compared with passage-2, day-7 cultured fetal RPE cells (Table 12). This comparison was undertaken for the cell subtypes cultured on BCE-ECM-coated dishes only since adult RPE cells were not cultured on TCTP dishes due to poor attachment and growth on this substrate. The overall detection frequencies of the secreted trophic factors in adult vs. fetal RPE-CM were similar; therefore, it is unlikely that the significant differences identified between cultured adult and fetal RPE cells were biased by detection frequencies (Table 11).

TABLE 12
Trophic factor composition of conditioned media collected from cultured
primary adult RPE cells and passage-2, day-7 cultured fetal RPE cells grown
on bovine corneal endothelial cell-extracellular matrix-coated dishes.

| | Mean (SEM) pg/μg RPE protein | | | | | | | | |
|---|---|---|---|---|---|---|---|---|---|
| | LIF | bFGF | HB-EGF | HGF | VEGF-A | NGF | BDNF | CNTF | PEDF |
| P2D7-fRPE | 8.2 | 92.8 | 149 | 521.4 | 19719.5 | 14.6 | 321.9 | 35 | 803526 |
| (n = 5) | (2.9) | (18.9) | (16.3) | (63.2) | (1540.8) | (3.4) | (37.0) | (5.4) | (121280) |
| Adult RPE | 135.9 | 308.3 | 366 | 605.9 | 9434.4 | 93.8 | 60.2 | 307.2 | 180381 |
| (n = 7) | (17.7) | (34.2) | (156.1) | (154.7) | (2147.6) | (14.8) | (5.6) | (118.0) | (77460) |
| p-value* | 0.003 | <0.001 | NS | NS | 0.005 | 0.003 | 0.036 | NS | 0.001 |

Abbreviations:
P2D7-fRPE, passage-2 day-7 fetal RPE;
LIF, leukemia inhibitory factor,
bFGF, basic fibroblast growth factor;
HB-EGF, heparin binding-epidermal growth factor;
HGF, hepatocyte growth factor;
VEGF-A, vascular endothelial growth factor-A;
NGF, neuronal growth factor;
BDNF, brain-derived neurotrophic factor;
CNTF, ciliary neurotrophic factor;
PEDF, pigment epithelium-derived factor;
NS, not significant.
*Unpaired t-test.

Preservation of Porcine Retina

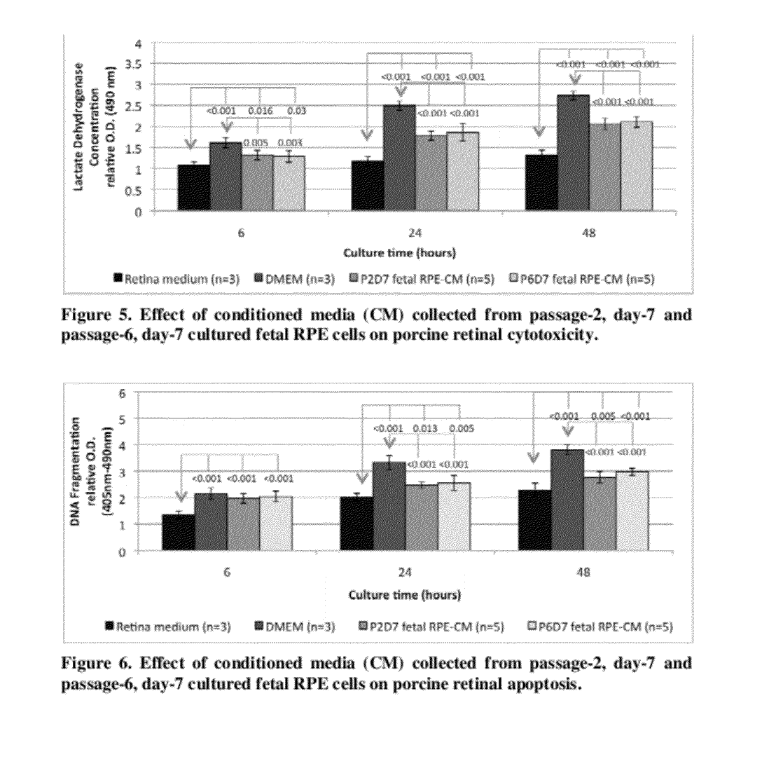

To determine if CM from cultured adult and fetal preparations affect retinal preservation to different degrees, six millimeter in diameter retinal explants were isolated from porcine eyes and cultured from 1-48 hours in retina medium (positive control), DMEM (negative control), and cultured RPE-CM (passage-2, day-7 and passage-6, day-7 cultured fetal RPE-CM and primary adult RPE-CM grown on BCE-ECM-coated dishes). LDH and DNA fragmentation were measured at each time point with the 1-hour results serving as the reference time point. At each time point, retinae in passage-2, day-7 and passage-6, day-7 cultured fetal RPE-CM showed significantly lower survival than the retinae in retina medium, significantly better survival than retinae in DMEM, and did not differ significantly from one another (FIGS. 5 and 6).

FIG. 5 illustrates the effect of conditioned media (CM) collected from passage-2, day-7 and passage-6, day-7 cultured fetal RPE cells on porcine retinal cytotoxicity. Porcine retina was cultured in CM collected from passage-2, day-7 (n=5; P2D7 fetal RPE-CM) and passage-6, day-7 (n=5; P6D7 fetal RPE-CM) cultured fetal RPE cells and in positive (n=3; retina medium) and negative (n=3; DMEM) controls. Mean±SEM lactate dehydrogenase concentration was compared for statistical significance (p<0.05) using a one-way ANOVA followed by a Holm-Sidak method for all pairwise multiple comparison.

FIG. 6 illustrates the effect of conditioned media (CM) collected from passage-2, day-7 and passage-6, day-7 cultured fetal RPE cells on porcine retinal apoptosis. Porcine retina was cultured in CM collected from passage-2, day-7 (n=5; P2D7 fetal RPE-CM) and passage-6, day-7 (n=5; P6D7 fetal RPE-CM) cultured fetal RPE cells and in positive (n=3; retina medium) and negative (n=3; DMEM) controls. Mean±SEM DNA fragmentation was compared for statistical significance (p<0.05) using a one-way ANOVA followed by a Holm-Sidak method for all pairwise multiple comparison.

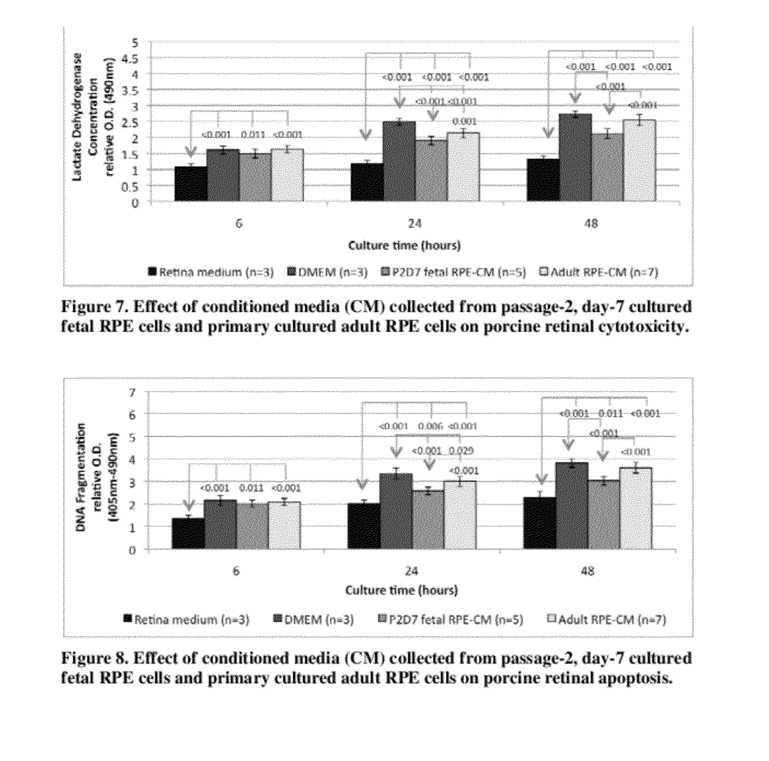

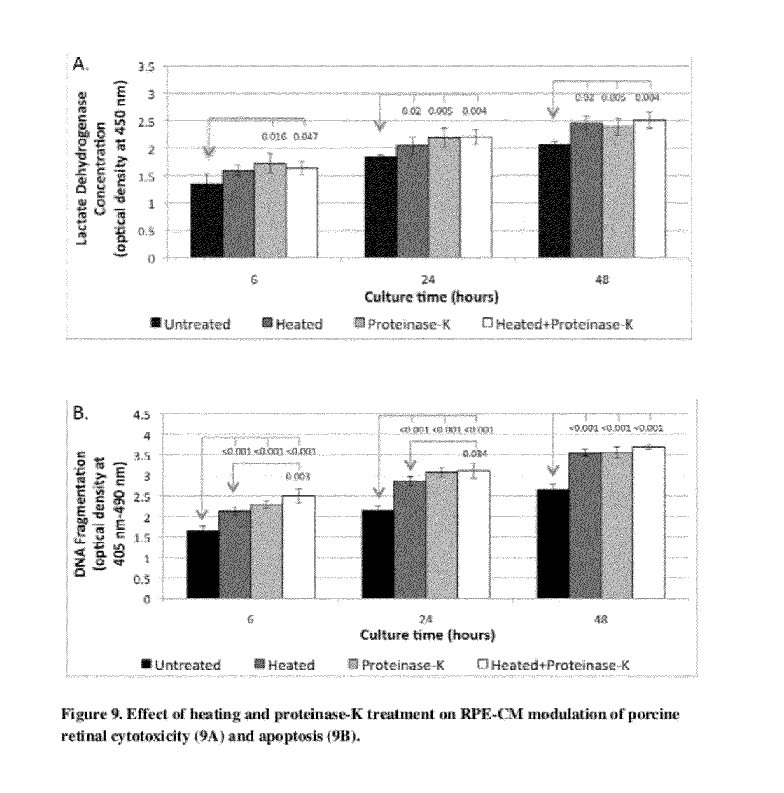

Passage-2, day-7 cultured fetal RPE-CM was significantly better that primary cultured adult RPE-CM at reducing retinal cytotoxicity and apoptosis at the 24- and 48-hour time points (FIGS. 7 and 8).

FIG. 7 illustrates the effect of conditioned media (CM) collected from passage-2, day-7 cultured fetal RPE cells and primary cultured adult RPE cells on porcine retinal cytotoxicity. Porcine retina was cultured in CM collected from passage-2, day-7 (n=5; P2D7 fetal RPE-CM) and cultured adult RPE cells (n=7; adult RPE-CM) and in positive (n=3; retina medium) and negative (n=3; DMEM) controls. Mean±SEM lactate dehydrogenase concentration was compared for statistical significance (p<0.05) using a one-way ANOVA followed by a Holm-Sidak method for all pairwise multiple comparison.

FIG. 8 illustrates the effect of conditioned media (CM) collected from passage-2, day-7 cultured fetal RPE cells and primary cultured adult RPE cells on porcine retinal apoptosis. Porcine retina was cultured in CM collected from passage-2, day-7 (n=5; P2D7 fetal RPE-CM) and cultured adult RPE cells (n=7; adult RPE-CM) and in positive (n=3; retina medium) and negative (n=3; DMEM) controls. Mean±SEM DNA fragmentation was compared for statistical significance (p<0.05) using a one-way ANOVA followed by a Holm-Sidak method for all pairwise multiple comparison.

In addition, by the 48-hour time point, retinal cytotoxicity and apoptosis in primary cultured adult RPE-CM did not differ significantly from that of DMEM.

Effect on Preservation of Porcine Retina

Porcine retinal cytotoxicity and apoptosis were compared after a 6-, 24- or 48-hour culture in one of two conditions: 1) passage-2, day-7 cultured fetal RPE-CM vs. co-culture with passage-2, day-7 fetal RPE cells, and 2) passage-6, day-7 cultured fetal RPE-CM vs. co-culture with passage-6, day-7 fetal RPE cells (Table 13). A significantly higher level of retinal cytotoxicity was identified in passage-2, day-7 and passage-6, day-7 cultured fetal RPE-CM than in the corresponding co-cultures at 24 and 48 hours. In addition, significantly more retinal apoptosis was identified in passage-2, day-7 cultured fetal RPE-CM than passage-2, day-7 cultured fetal RPE-retina co-culture at the 6-hour time point and in passage-6, day-7 cultured fetal RPE-CM than passage-6, day-7 cultured fetal RPE-retina co-culture at the 6- and 48-hour time points (Table 13).

TABLE 13
Effect of conditioned media collected from cultured fetal RPE cells or fetal RPE-
retina co-cultures on porcine retinal cytotoxicity and apoptosis.

| | Mean ± SEM (%) of Retina Medium | | | | | |
|---|---|---|---|---|---|---|
| Type of Conditioned | Retinal Cytotoxicity | | | Retinal Apoptosis | | |
| Medium (n = 5) | 6 hours | 24 hours | 48 hours | 6 hours | 24 hours | 48 hours |
| Passage-2, day-7 | 122.2 ± 6.0 | 150.8 ± 5.2* | 156.1 ± 5.9^{†} | 146.2 ± 7.6 | 122.6 ± 3.2 | 121.5 ± 5.4 |
| cultured fetal RPE | | | | | | |
| Passage-2, day-7 fetal | 118.8 ± 8.0 | 113.9 ± 5.9 | 111.6 ± 3.0 | 123.9 ± 6.9 | 118.1 ± 3.5 | 114.0 ± 2.1 |
| RPE-retina co-culture | | | | | | |
| Passage-6, day-7 | 119.5 ± 7.1 | 157.4 ± 10.0^{‡} | 160.0 ± 5.5^{§} | 151.5 ± 8.5 | 126.4 ± 8.1 | 130.5 ± 3.5 |
| cultured fetal RPE | | | | | | |
| Passage-6, day-7 fetal | 123.2 ± 5.8 | 118.5 ± 4.9 | 114.5 ± 3.7 | 128.3 ± 5.6 | 117.7 ± 4.0 | 116.1 ± 4.9 |
| RPE-retina co-culture | | | | | | |

Data are represented as amount (mean ± SEM) of extracellular LDH (‘retinal cytotoxicity’) and retinal DNA fragmentation (‘retinal apoptosis’) compared to levels found after retinal culture in the retina medium at the corresponding time points. Corresponding pairs (passage-2, day-7 fetal cells vs. retina co-culture and passage-6, day-7 fetal cells vs. retina co-culture) were evaluated for statistical significance (p < 0.05) at each time point by an un-paired t-test.
The following comparisons were statistically significant:
*p = 0.046,
^{†}p = 0.005,
^{‡}p = 0.02, and
^{§}p = 0.005.

Effect on Trophic Factor Production

In addition to quantifying retinal cytotoxicity and apoptosis, trophic factor composition of passage-2, day-7 fetal RPE-retina-CM (co-culture) was analyzed at the 24- and 48-hour time points via multiplex ELISA. This was performed on three fetal RPE cell lines (mean age, 20.7 weeks gestation) grown on BCE-ECM-coated dishes. In comparison to cultured fetal RPE-CM, at the 24-hour time point, the fetal RPE-retina co-culture CM had significantly higher levels of bFGF and HGF and, at the 48-hour time point, significantly higher levels of bFGF, HB-EGF, and HGF (Table 14). There was also a strong trend for increased BDNF production by the fetal RPE-retina co-culture at the 24-hour (p=0.057) and 48-hour (p=0.059) time points.

TABLE 14
Comparison of trophic factor composition of conditioned media collected
from passage-2, day-7 fetal RPE cells and a co-culture of passage-2, day-7
fetal RPE cells with porcine retina after 24 and 48 hours of culture.

| | Mean (SEM) pg/μg RPE protein | | | | |
|---|---|---|---|---|---|
| | 24-hour culture | | 48-hour culture | | |
| | bFGF | HGF | bFGF | HGF | HB-EGF |
| −retina (n = 3) | 139.1 (5.0) | 45.4 (10.6) | 74.2 (38.3) | 46.5 (23.1) | 332 (9.8) |
| +retina (n = 3) | 3036.1 (317.5) | 154 (11.3) | 1383.9 (254.4) | 230 (36.4) | 419.7 (20.5) |
| p-value* | <0.001 | 0.002 | 0.007 | 0.013 | 0.018 |

Abbreviations: bFGF, basic fibroblast growth factor; HGF, hepatocyte growth factor; HB-EGF, heparin binging-epidermal growth factor.
*Unpaired t-test.

TABLE 15
List of secreted proteins found in significantly different quantities from cultured adult vs. fetal RPE cells.

| | Accession | Size | | Fold Change | *p- |
|---|---|---|---|---|---|
| Identified Protein Name | number | (kDa) | General functions | (fetal/adult) | value |
| Cathepsin B | IPI00295741.4 | 38 | Regulation of apoptosis | 0.2 | 0 |
| Alpha-crystallin B chain | IPI00021369.1 | 20 | Negative regulation of cell growth; | 0.2 | 0 |
| | | | anti-apoptosis | | |
| Retinoid | IPI00029250 | 65 | Implicated in some types of Leber | 0.2 | 0 |
| isomerohydrolase (RPE- | | | Congenital Amaurosis and retinitis | | |
| 65) | | | pigmentosa; regulation of rhodopsin | | |
| | | | gene expression | | |
| Serpin peptidased | IPI00550991.3 | 51 | Increases cortical neuron apoptosis | 0.3 | 0 |
| inhibitor | | | | | |
| Superoxide dismutase | IPI00022314.1 | 25 | Negative regulation of neuron | 0.3 | 0 |
| | | | apoptosis; affects neuronal | | |
| | | | development | | |
| Prosaposin | IPI00012503.1 | 58 | Increases cell apoptosis (caspase- | 0.3 | 0.02 |
| | | | dependent) | | |
| Metalloproteinase | IPI00032292.1 | 23 | Anti-apoptotic; regulates cell | 0.3 | 0 |
| inhibitor 1 (TIMP) | | | proliferation | | |
| Secretogranin-2 | IPI00009362.2 | 71 | Negative regulation of cell | 0.3 | 0 |
| | | | apoptosis; MAPKKK cascade; cell | | |
| | | | proliferation, migration | | |
| Granulin (isoform 1) | IPI00296713.4 | 64 | Cytokine/growth factor activity; | 0.3 | 0.02 |
| | | | positive regulation of epithelial cell | | |
| | | | proliferation | | |
| C3 Complement | IPI00783987.2 | 188 | Complement system activation | 0.3 | 0 |
| (fragment) | | | (classical and alternative pathways) | | |
| Alpha-2-macroglobulin | IPI00478003.2 | 163 | Protease inhibitor and cytokine | 0.3 | 0 |
| | | | transporter; negative regulation of | | |
| | | | complement activation | | |
| NAD(P)H dehydrogenase | IPI00012069 | 31 | Positive regulation of neuron | 0.3 | 0 |
| | | | apoptosis; oxidation reduction | | |
| Insulin-like growth factor- | IPI00018305 | 32 | Cell growth, proliferation, | 0.3 | 0.01 |
| binding protein-3 | | | differentiation, survival; positive | | |
| | | | regulation of apoptosis | | |
| Retinaldehyde-binding | IPI00218633.5 | 36 | Implicated in retinitis pigmentosa | 0.3 | 0 |
| protein-1 | | | and rod-cone dystrophies; 11-cis- | | |
| | | | retinal transport; visual cycle | | |
| Galectin-1 | IPI00219219.3 | 15 | Autocrine negative growth factor; | 0.3 | 0 |
| | | | modulates cell-cell, cell-matrix | | |
| | | | interactions; regulation of apoptosis; | | |
| | | | positive regulation of I-kappaB | | |
| | | | kinase/NF-kappaB cascade | | |
| Inter-alpha (globulin) | IPI00218192 | 101 | Acute phase response; cell apoptosis | 0.3 | 0 |
| inhibitor H4 (isoform 2) | | | | | |
| Ceruloplasmin | IPI00017601.1 | 122 | Copper ion transport; cellular iron | 0.4 | 0 |
| | | | ion homeostasis; oxidation reduction | | |
| Collagen alpha-2(I) chain | IPI00304962 | 129 | Transforming growth factor beta | 0.4 | 0 |
| | | | receptor signaling pathway; Rho | | |
| | | | protein signal transduction; cell | | |
| | | | migration, proliferation, and growth | | |
| Latent-transforming | IPI00292150.4 | 195 | Transforming growth factor beta | 0.4 | 0 |
| growth factor beta-binding | | | receptor signaling pathway; protein | | |
| protein-2 | | | secretion | | |
| Extracellular matrix | IPI00003351.2 | 61 | Positive regulation of I-kappaB | 0.4 | 0.02 |
| protein-1 (isoform 1) | | | kinase/NF-kappaB cascade; cell | | |
| | | | growth, apoptosis, and proliferation | | |
| Glutathione peroxidase-3 | IPI00026199.2 | 26 | Cell damage, survival, viability, | 0.4 | 0 |
| | | | growth, and apoptosis; response to | | |
| | | | oxidative stress | | |
| Thrombospondin-1 | IPI00296099.6 | 129 | Activation of MAPK activity; | 0.5 | 0 |
| | | | fibronectin, integrin, bFGF, and | | |
| | | | TGF-beta binding; increases neurite | | |
| | | | outgrowth | | |
| Fibronectin (isoform 1) | IPI00022418.1 | 263 | Cell adhesion, migration, spreading, | 0.5 | 0 |
| | | | and apoptosis | | |
| Inter-alpha (globulin) | IPI00305461 | 106 | Cell apoptosis; extracellular matrix | 0.5 | 0 |
| inhibitor H2 | | | stabilization | | |
| Low-density lipoprotein | IPI00020557.1 | 505 | Positive regulation of anti-apoptosis; | 0.5 | 0 |
| related protein-1 (alpha-2- | | | cell proliferation, attachment, and | | |
| macroglobulin receptor) | | | death | | |
| EGF-containing fibulin- | IPI00029658.1 | 55 | Visual perception; VEGF-A | 0.5 | 0.01 |
| like extracellular matrix | | | regulation | | |
| protein-1 (isoform 1) | | | | | |
| Glutathione S-transferase | IPI00019755.3 | 28 | Cell redox homeostasis; stress | 0.5 | 0.02 |
| omega-1 | | | response | | |
| Semaphorin-3B (isoform | IPI00012283.2 | 83 | Axonal (growth cone) guidance; | 0.5 | 0.01 |
| 1) | | | apoptosis inducer | | |
| Laminin subunit alpha-5 | IPI00783665.4 | 400 | Cell adhesion, survival, apoptosis, | 0.5 | 0 |
| | | | migration, and proliferation | | |
| Fibulin-1 (isoform B) | IPI00218803 | 77 | Cell apoptosis; extracellular matrix | 0.6 | 0.01 |
| | | | structural constituent; Ca⁺² ion | | |
| | | | binding; implicated in AMD | | |
| Superoxide dismutase | IPI00027827 | 26 | Caspase-dependent apoptosis; | 0.6 | 0.02 |
| (Cu—Zn) | | | decreases apoptosis induced by | | |
| | | | NGF-depletion | | |
| Protein kinase C-binding | IPI00015260.1 | 91 | Regulation of neural cell growth and | 0.6 | 0.01 |
| protein NELL2 | | | differentiation; cell adhesion | | |
| Heat shock protein beta-1 | IPI00025512.2 | 23 | Anti-apoptotic (regulates CASP3, 8, | 0.6 | 0.03 |
| (cytoplasm/nucleus) | | | 9, P38 MAPK, Jnk, NFkB, Erk, Akt, | | |
| | | | IKBKB) | | |
| Insulin-like growth factor- | IPI00020996.5 | 66 | Insulin-like growth factor binding | 0.6 | 0.03 |
| binding protein complex | | | | | |
| (acid labile subunit) | | | | | |
| Tumor protein | IPI00009943 | 21 | Cell proliferation; apoptosis | 0.7 | 0.05 |
| (translationally-controlled- | | | | | |
| 1) | | | | | |
| Transforming growth | IPI00018219.1 | 68 | Negative regulation of cell adhesion; | 0.7 | 0.01 |
| factor (beta-induced) | | | cell proliferation; extracellular | | |
| | | | matrix organization | | |
| Complement C4 (acidic | IPI00032258.4 | 193 | Classical activation pathway | 0.7 | 0.01 |
| form) | | | | | |
| Galectin-3-binding protein | IPI00023673.1 | 65 | Cell adhesion and apoptosis | 1.5 | 0.03 |
| Collagen alpha-1(VI) | IPI00291136 | 109 | Cell growth and apoptosis; platelet- | 1.6 | 0.02 |
| chain | | | derived growth factor binding | | |
| Collagen alpha-1(XI) | IPI00218539 | 182 | Cell adhesion, organization, and | 1.8 | 0.02 |
| chain (isoform b) | | | development | | |
| Inter-alpha (globulin) | IPI00328829 | 106 | Extracellular matrix stabilization; | 2.1 | 0.02 |
| inhibitor H5 | | | cell apoptosis | | |
| Hepatoma-derived growth | IPI00020956.1 | 27 | Cell proliferation; signal | 2.1 | 0.04 |
| factor | | | transduction; heparin binding | | |
| Gelsolin (isoform 1) | IPI00026314.1 | 86 | Cell adhesion and apoptosis | 2.1 | 0.01 |
| Pigment epithelium- | IPI00006114.4 | 46 | Neurotrophic; affects NPD1 | 2.5 | 0 |
| derived factor | | | synthesis, secretion; increases NGF, | | |
| | | | GDNF, and BDNF mRNA | | |
| | | | expression (in rats) | | |
| Collectin-12 (isoform 1) | IPI00414467.6 | 82 | Removes oxidized or apoptotic cells | 2.5 | 0.01 |
| (transmembrane receptor) | | | by recognizing oxidized | | |
| | | | phospholipids | | |
| Cathepsin L2 (lysosome) | IPI00000013.1 | 37 | Regulation of cell apoptosis | 2.6 | 0 |
| Collagen alpha-1(XVIII) | IPI00022822.5 | 154 | Cell adhesion, proliferation, and | 2.8 | 0 |
| chain (isoform 2) | | | migration; extracellular matrix | | |
| | | | organization; positive regulation of | | |
| | | | apoptosis | | |

*2-tailed t-test.

Retinal Trophic Factor Production

To determine whether trophic factor secretion by the porcine retina could have accounted for the significant differences in CM collected from fetal RPE cells vs. fetal RPE-retina co-culture (Table 14), porcine retinal trophic factor secretion was quantified by multiplex ELISA. After accounting for non-specific binding, the only factors identified in the retina-CM were bFGF, HB-EGF, and VEGF-A. After 48 hours of culture in DMEM, the porcine retina produced 21-30 pg of VEGF-A and 25-36 pg of HB-EGF per explant. The amount of bFGF increased from 98 pg/explant at the 1-hour to 128 pg/explant at the 6-hour time point and remained relatively constant thereafter throughout the 48-hour culture period. The relative contribution of retinal VEGF-A, HB-EGF, and bFGF to the amounts found in the fetal RPE-retina co-culture CM after 24 and 48 hours of culture were approximately 0%, 4-8%, and 4-9%, respectively.

Retinal Trophic Factor mRNA Expression

Compared to time 0, there was no transcript upregulation after 24 or 48 hours of culture that would correspond to the significantly increased levels of selected proteins identified in co-culture CM. In addition, while retinal VEGF-A protein levels were almost identical to HB-EGF and were 3-5 times lower than those of bFGF, the amount of VEGF-A mRNA transcript was approximately three orders of magnitude higher than those of HB-EGF and bFGF.

iTRAQ

A total of 381 proteins were identified with at least two peptides with a confidence interval of ≧95%. False discovery rate of peptides was estimated at 4.7%. Of these 381 proteins, 95 (25%) were significantly (p<0.05) different between cultured adult RPE-CM and passage-2, day-7 cultured fetal RPE-CM (cells grown on BCE-ECM-coated dishes). Forty-seven (49%) of these significantly different proteins, the vast majority of which were secreted proteins, previously have been shown to regulate apoptosis, affect cellular response to oxidative stress, or involve the complement cascade (Table 15). Candidate proteins that seem most likely to have contributed to the differential effect of cultured adult RPE-CM and passage-2, day-7 cultured fetal RPE-CM on retinal preservation are listed in Table 16. These proteins were identified as ‘candidate proteins’ based on their function and the degree of the statistical difference between adult and fetal CM. Fetal RPE cells secreted significantly less IGFBP-3 (relative ratio=0.3), semaphorin-3B (relative ratio=0.5), and transforming growth factor-beta (TGF-β) (relative-ratio=0.7), and significantly more hepatoma-derived growth factor (HDGF) (relative ratio=2.1) and gelsolin (relative ratio=2.1). iTRAQ confirmed significantly higher production of PEDF (relative ratio=2.5) by fetal RPE cells which was previously shown by multiplex ELISA (Table 11).

TABLE 16
iTRAQ-identified proteins with a potential
to affect retinal preservation.⁵¹⁻⁵⁸

| | | Mean ratio | |
|---|---|---|---|
| Protein Name | kDa | (fetal/adult) | p-value* |
| Insulin-like growth factor-binding | 32 | 0.3 | 0.01 |
| protein-3 | | | |
| Semaphorin-3B (isoform 1) | 83 | 0.5 | 0.01 |
| Transforming growth factor (beta- | 68 | 0.7 | 0.01 |
| induced) | | | |
| Hepatoma-derived growth factor | 27 | 2.1 | 0.04 |
| Gelsolin (isoform 1) | 86 | 2.1 | 0.01 |
| Pigment epithelium-derived factor | 46 | 2.5 | <0.01 |

*2-tailed t-test.

Trophic Factor Receptor Occupancy

Compared to the CM isolated from passage-2, day-7 fetal RPE cells, the concentrations as well as the corresponding trophic factor receptor occupancies for VEGF-A and PEDF were significantly lower in the CM isolated from cultured adult RPE cells and passage-6, day-7 fetal RPE cells (Table 17).

TABLE 17
Trophic factor concentrations and receptor occupancies.

| | Trophic Factor Concentration (pM), Mean ± | | |
|---|---|---|---|
| Trophic | SEM (% Occupancy, Mean ± SEM) | | |
| Factor | Adult | P2D7 fetal | P6D7 fetal |
| (K_{D} [pM]) | (n = 7) | (n = 5) | (n = 5) |
| LIF (2000) | 2.1 ± 0.5 | 0.7 ± 0.5 | 0.4 ± 0.1 |
| | (0.1 ± 0.02) | (0.04 ± 0.02) | (0.02 ± 0.005) |
| bFGF (1000) | 5.1 ± 1.0 | 3.1 ± 0.9 | 8.2 ± 2.3 |
| | (0.5 ± 0.1) | (0.3 ± 0.09) | (0.8 ± 0.2) |
| HB-EGF (200) | 4.3 ± 1.5 | 4.3 ± 2.2 | 2.7 ± 1.0 |
| | (2.1 ± 0.7) | (2.1 ± 1.1) | (1.3 ± 0.5) |
| HGF (100) | 1.8 ± 0.5 | 4.3 ± 1.9 | 1.7 ± 1.0 |
| | (1.7 ± 0.5) | (4.1 ± 1.9) | (1.7 ± 1.0) |
| VEGF-A (1000) | 47.7 ± 4.5* | 364.3 ± 64.4 | 132.5 ± 19.1^{†} |
| | (4.6 ± 0.4) | (26.7 ± 6.0) | (11.7 ± 1.9) |
| NGF (300) | 2.3 ± 0.6 | 0.4 ± 0.1 | 1.6 ± 0.7 |
| | (0.8 ± 0.2) | (0.1 ± 0.04) | (0.5 ± 0.2) |
| BDNF (1000) | 1.5 ± 0.2 | 7.1 ± 4.1 | 3.8 ± 1.4 |
| | (0.2 ± 0.02) | (0.7 ± 0.4) | (0.4 ± 0.1) |
| CNTF (100) | 5.0 ± 2.1 | 3.4 ± 2.5 | 2.8 ± 1.8 |
| | (0.3 ± 0.1) | (3.3 ± 2.5) | (2.7 ± 1.8) |
| PEDF (3000) | 656.2 ± 154.3^{‡} | 27012 ± 14810 | 14061 ± 11792^{§} |
| | (17.9 ± 4.9) | (90.0 ± 83.2) | (82.4 ± 79.7) |

Abbreviations: P2D7, passage-2 day-7; P6D7, passage-6 day-7; pM, picomoles; LIF, leukemia inhibitory factor, bFGF, basic fibroblast growth factor; HB-EGF, heparin binding-epidermal growth factor; HGF, hepatocyte growth factor; VEGF-A, vascular endothelial growth factor-A; NGF, neuronal growth factor; BDNF, brain-derived neurotrophic factor; CNTF, ciliary neurotrophic factor; PEDF, pigment epithelium-derived factor. Significantly different (t-test) from passage-2 day-7 fetal RPE at
*^{,†,‡,§}p < 0.05.

### Example 3

### Methods

Conditioned Media

Human fetal eyes (17-22 weeks gestation) were obtained through Advanced Bioscience Resources, Inc (ABR; Alameda, Calif.). Fetal RPE was isolated after incubation in 0.8 mg/ml collagenase IV (Sigma-Aldrich, St. Louis, Mo.) and cultured on uncoated tissue culture dishes as previously described in, Kolomeyer A M, Sugino I K, Zarbin M A. Invest Ophthalmol Vis Sci 2011; 52:5973-5986. 8RPE culture purity was verified with immunocytochemistry staining for pan-cytokeratin (Sigma-Aldrich).

For RPE-CM collection, passage-2 RPE cells were seeded at 1.2×10⁶ cells/well onto 12-well plates (Costar; Corning Inc., Corning, N.Y.). Seven days post-passage cultures (passage-2, day-7; n=3) were thoroughly washed with Dulbecco's Phosphate Buffered Saline (DPBS) to remove any remnants of the RPE medium (specifically FBS and bFGF), and incubated in two ml of Dulbecco's Modified Eagle Medium (DMEM) for 24 hours at 37° C., 10% CO₂. After collection, RPE-CM was centrifuged to remove any cellular debris and supernatant frozen and stored at −80° C. Trophic factor composition (expressed as pg per μg of fetal RPE protein) was verified via multiplex ELISA (Aushon Biosystems, Woburn, Mass.) to confirm whether the secretion profile was similar to that previously reported.

Experimental Design

Porcine donor eyes (obtained from a local abattoir within three hours of enucleation) and human donor eyes (obtained from the Lions Eye Institute for Transplant and Research [Tampa, Fla.] and eye banks placing tissue through the National Disease Research Interchange [Philadelphia, Pa.]) were prepared for dissection. Six-millimeter trephine blades (Storz Ophthalmic-Bausch and Lomb, Manchester, Mo.) were used to isolate five to six equatorial, full-thickness retina tissue explants (avoiding the peripapillary region), which were separated gently from the RPE-Bruch's membrane-choroid-sclera. Explants were cultured on autoclaved filter paper in wells of 96 well plates containing one of three media at 37° C., 10% CO₂: 1) retina medium [positive control; DMEM supplemented with 10% FBS, 0.2 mg/ml glutamine (Gibco-Invitrogen), 2.0 mM ascorbic acid (Sigma-Aldrich), 0.1 mM taurine (Sigma-Aldrich), 10 μg/ml porcine insulin (Sigma-Aldrich), 1 mM pyruvate (Sigma-Aldrich), 250 μg/ml Amphotericin B (Gibco-Invitrogen), and 50 mg/ml gentamicin sulfate (Cellgro-Mediatech Inc.)]; 2) DMEM (negative control); or 3) RPE-CM.

Conditioned Medium Assessment

Protein Degradation and Digestion

With the intention of assessing whether the retinal preservation activity is protein-based, heating and/or proteinase-k digestion were used to degrade the protein component in RPE-CM. Heating was performed at 80° C. for 15 minutes in a water bath (VWR Scientific, West Chester, Pa.) after which the medium was centrifuged for 10 minutes at 10,000 rpm, 4° C. to separate out the denatured proteins, and supernatant frozen at −80° C. Untreated medium was likewise centrifuged and stored. Proteinase-K-agarose (Sigma-Aldrich) was washed with autoclaved water. Untreated and heat-treated CM were then added to the proteinase-K-agarose complex, incubated at 37° C. for 80 minutes (shaken every 10 minutes), centrifuged at 4,000 rpm for one minute, and supernatant frozen at −80° C. Sepharose beads alone were also mixed with the untreated and heat-treated CM as a control to determine non-specific binding.

RPE-CM Preparations of Different Concentrations

RPE-CM was diluted to 10%, 20%, and 50% with DMEM or concentrated to 200% or 500% using a 3-kDa molecular cut-off filter (Amicon-Millipore, Danvers, Mass.). The filter was pre-coated with 5% BSA (Sigma-Aldrich) in DPBS for 30 minutes at room temperature in order to reduce non-specific binding and rinsed with DPBS. The prepared CM was frozen at −80° C.

Fractionation:

A 100-kDa molecular cut off filter followed by a 3-kDa filter (Amicon-Millipore) was used to isolate the 3-100 kDa sub-fraction of RPE-CM. The filters were washed with BSA (Sigma-Aldrich) and rinsed with DPBS. One ml of RPE-CM was centrifuged through the 100-kDa filter for ˜8 minutes at 4,000 g, 4° C. until ˜100 μl was left in the column. DMEM was flushed through the filter to maximize isolation of factors <100-kDa. The flow through (<100-kDa) was centrifuged through the 3-kDa filter for ˜25 minutes at 4,000 g until ˜100 μl was left (3-100 kDa). As an additional rinse, one ml of DMEM was added to the filter and spun through until ˜100 μl was left in the column. The final volume was brought up to one ml with DMEM and frozen at −80° C.

Recombinant Proteins and Neutralizing Antibodies

Recombinant human proteins included PEDF (BioProductsMD, Middletown, Md.), HGF (R&D Systems, Minneapolis, Minn.), and VEGF-A (R&D Systems, Minneapolis, Minn.). Neutralizing antibodies included anti-PEDF (1.0 μg/ml; BioProductsMD, Middletown, Md.), anti-HGF (100 μg/ml; R&D Systems, Minneapolis, Minn.), and anti-VEGF (10 μg/ml; R&D Systems, Minneapolis, Minn.). IgG control (1 mg/ml; R&D Systems, Minneapolis, Minn.) was used to account for potential non-specific binding. Concentrations of neutralizing antibodies used in all experiments were based on the manufacturer's recommended dose and those previously shown in the literature to achieve maximal blockade of protein activity. PEDF neutralizing antibody was incubated with RPE-CM for 30 minutes at 37° C. prior to immersing porcine retina tissue. The HGF and VEGF-A neutralizing antibodies as well as control IgG antibody was incubated with RPE-CM for one hour at room temperature prior to immersing porcine retinal tissue. Porcine retinal explants were cultured for 1, 24, and 48 hours in one of the following conditions: 1) DMEM (n=5); 2) RPE-CM (n=5); 3) DMEM+peptide concentration yielding 50% receptor occupancy for PEDF (n=5); 4) DMEM+peptide concentration yielding 50% receptor occupancy for HGF (n=5); 5) DMEM+peptide concentration yielding 50% receptor occupancy for VEGF-A (n=5); 6) DMEM+peptide concentration yielding 90% receptor occupancy for PEDF (n=9); 7) DMEM+peptide concentration yielding 90% receptor occupancy for HGF (n=9); 8) DMEM+peptide concentration yielding 90% receptor occupancy for VEGF-A (n=5); 9) RPE-CM+PEDF neutralizing antibody (n=7); 10) RPE-CM+HGF neutralizing antibody (n=7); 11) RPE-CM+PEDF neutralizing antibody+HGF neutralizing antibody (n=7); and 12) RPE-CM+IgG control (n=5). Trophic factor concentrations required to achieve specific % receptor occupancy were calculated based on equations previously described (Table 18). Retinal cytotoxicity and amount of DNA fragmentation was assessed at these time points as described in the Analysis of RPE-CM effectiveness section. The data are expressed relative to the 1-hour levels and as percent of DMEM culture levels.

TABLE 18
Trophic factor dissociation constant, concentration,
and corresponding percent receptor occupancy.

| | | Concentration (pg/μl) required | | |
|---|---|---|---|---|
| Trophic | K_{D} | to achieve % receptor occupancy | | |
| Factor | (pM) | 10% | 50% | 90% |
| PEDF | 3000 | 313 | 3000 | 27000 |
| HGF | 100 | 11 | 100 | 900 |
| VEGF-A | 1000 | 111 | 1000 | 9000 |

Based on known the known dissociation constant (K_{D}) for each trophic factor, the concentration of recombinant protein necessary to achieve a particular percent receptor occupancy (i.e., 10%, 50%, or 90%) was calculated using the following formula that we have described previously, ^{7, 8} Receptor occupancy = [L]/[K_{D} + L], where L—ligand concentration and K_{D}—dissociation constant.

Aged-Human Retina

Retinal tissue was harvested from Caucasian non-AMD (n=6; mean±SD age, 74.7±6.8 years; range, 63-83 years) and AMD cadaveric eyes (n=6; 75.7±9.5 years; range, 62-81 years), and from non-AMD African American (n=6; 66.5±11.9 years; range 45-77 years) cadaveric eyes (Table 19). Six retinal explants were taken from each eye; two each were cultured in one of the three media (same as for porcine eyes as outlined in the Experimental Design section). For each pair of eyes, explants from one eye were cultured for 24 hours and from the other for 48 hours in the above-mentioned conditions.

TABLE 19
Human retinal donor information.

| | | | D to P | D to R |
|---|---|---|---|---|
| | A/G/R | Ocular Pathology | (h:m) | (h:m) |
| 1 | 75/M/AA | a: hard and soft, small and intermediate-size | 4:32 | 46:25 |
| | | macular and perimacular drusen; equatorial small, | | |
| | | hard and confluent soft, intermediate-size drusen; | | |
| | | hard exudates/hemorrhage near the optic nerve; | | |
| | | b: central macular RPE defect; equatorial small, | | |
| | | hard and confluent soft, intermediate-size drusen | | |
| 2 | 72/F/C | a, b: few small, hard peripheral drusen | 5:34 | 31:30 |
| 3 | 63/M/C | a: small, central macular druse; small, hard | 5:05 | 42:55 |
| | | peripheral drusen; b: small, hard peripheral druse | | |
| 4 | 80/M/C | a: small, hard macular drusen and two large | 4:17 | 28:30 |
| | | perimacular drusen with RPE hyper-pigmentation; | | |
| | | many peripheral drusen; b: small macular drusen; | | |
| | | one intermediate and large perimacular druse; | | |
| | | speckled RPE hyperplasia with peripheral drusen | | |
| 5 | 75/F/C | a, b: small macular drusen; small, hard peripheral | 5:53 | 36:40 |
| | | drusen; peripapillary choroidal hyperpigmentation | | |
| 6 | 77/F/AA | a: few small, hard macular and posterior pole | 4:25 | 30:05 |
| | | drusen; b: small macular druse; small, hard drusen | | |
| | | in posterior pole | | |
| 7 | 71/M/C | Diagnosed dry AMD (OS > OD) in 2006 | 5:55 | 46:00 |
| | | a: extramacular geographic atrophy, extensive, | | |
| | | small and intermediate-size macular drusen; b: RPE | | |
| | | hyperpigmentation associated with small macular | | |
| | | drusen; possible choroidal neovascular membrane | | |
| 8 | 80/M/C | a: large extramacular drusen with RPE defect; few | 4:30 | 31:35 |
| | | small macular, perimacular, and peripheral drusen; | | |
| | | peripapillary hyperpigmentation; b: few small | | |
| | | macular drusen; perimacular, intermediate-size | | |
| | | hard drusen | | |
| 9 | 89/M/C | Diagnosed neovascular AMD in 2004-2005 (treated | 5:43 | 46:56 |
| | | with ranibizumab) | | |
| | | a: confluent, intermediate-size macular soft drusen | | |
| | | with RPE hyperplasia; possible epiretinal | | |
| | | membrane; b: RPE hyperplasia with associated | | |
| | | soft, intermediate-size drusen (possible epiretinal | | |
| | | membrane) | | |
| 10 | 62/M/C | AMD donor (as per family) | 4:19 | 44:51 |
| | | a, b: small, hard macular drusen; possible choroidal | | |
| | | neovascular membrane; perimacular RPE | | |
| | | hyperplasia with small, hard drusen; small, hard | | |
| | | peripheral drusen | | |
| 11 | 45/M/AA | a: small, hard macular druse; small, hard posterior | 4:45 | 27:31 |
| | | pole drusen; b: small and intermediate-size, hard | | |
| | | posterior pole drusen | | |
| 12 | 78/M/C | a: intermediate and large, hard macular drusen; | 4:02 | 37:54 |
| | | many small, hard peripheral drusen with RPE | | |
| | | hyperplasia; large, hard druse along the equator; | | |
| | | b: intermediate- and large-size, hard macular | | |
| | | drusen; laser in temporal periphery; large, hard | | |
| | | equatorial druse | | |
| 13 | 74/F/AA | a: small, hard macular drusen and possible scar; | 5:24 | 34:24 |
| | | few small, hard peripheral drusen; b: small, hard | | |
| | | and intermediate-size macular drusen; calcified | | |
| | | deposit on optic nerve | | |
| 14 | 64/F/AA | a, b: no pathology | 5:44 | 38:34 |
| 15 | 64/M/AA | a, b: possible macular RPE defect; small, hard | 5:14 | 37:35 |
| | | peripheral drusen | | |
| 16 | 69/M/C | a: small, hard macular drusen; mixed-size | 3:20 | 37:50 |
| | | peripheral drusen; b: perimacular hard drusen; | | |
| | | mixed-size peripheral drusen | | |
| 17 | 83/F/C | a: small, soft macular drusen; few small, hard | 4:32 | 21:58 |
| | | peripheral drusen, laser spots (nasal to the optic | | |
| | | nerve); b: small, soft macular drusen, few | | |
| | | peripheral drusen (>a) | | |
| 18 | 81/F/C | AMD (treated with unspecified injections, 2007-8) | 4:52 | 36:22 |
| | | a: macular retinal scar; b: small and intermediate- | | |
| | | sized, hard macular drusen; some peripheral small, | | |
| | | hard drusen | | |

Abbreviations: A/G/R, age/gender/race; M, male; F, female; AA, African American; C, Caucasian; a and b, randomly assigned fellow eyes; D-P, death to preservation; D-R, death to receipt; h:m, hours:minutes.

Analysis of RPE-CM Effectiveness

To assess retinal cytotoxicity, a lactate dehydrogenase (LDH) in vitro toxicology assay (TOX-7; Sigma-Aldrich) was used to determine the effects of RPE-CM following one of the above manipulations on retinal membrane integrity as previously described in Kolomeyer A M, et. al. Invest Ophthalmol Vis Sci 2011; 52:5973-5986. Media from three retinal explants from three non-paired eyes were collected after 1, 6, 24, and 48 hours incubation in treated media and centrifuged to remove cellular debris. The supernatant was frozen at −20° C. and processed as per manufacturer's instructions. An absorbance microplate reader (ELx800, BioTek, Winooski, Vt.) allowed assessment of colorimetric absorbances at 450 nm. The data are expressed relative to the 1-hour levels.

To determine presence and amount of retinal cell apoptosis, cell death detection ELISA assay kit (Roche Diagnostics, Piscataway, N.J.) was used to quantify the effects of treated RPE-CM on the amount of retinal DNA fragmentation as previously described. Briefly, after 1, 6, 24, or 48 hours of culture in treated RPE-CM (three retinal explants from three non-paired eyes), the explants were homogenized in 200 μl of provided lysis buffer by trituration and processed as per manufacturer's instructions. Absorbances were measured at 405 nm with a reference wavelength at 490 nm (ELx800, BioTek). The data are expressed relative to the 1-hour levels.

Confocal Analysis

For time zero controls, one explant from four different eyes was fixed in 4% paraformaldehyde immediately after detachment from the RPE. The remaining explants from six porcine eyes (three eyes per time point) were cultured in one of the three media for 24 or 48 hours. The explants were placed in fixative for 24 hours at 4° C. A lab member randomized the explants using a random sequence generator function (www.random.org). Following rinsing in DPBS, explants were embedded in 4% agar (Fluka; Sigma-Aldrich) and vibratomed (VIBRATOME Series 1000, Bannockburn, Ill.) into 70-micron-thick cross sections, which were transferred to ProbeOn Plus slides (Thermo Fisher Scientific). Following washing with DPBS, sections were incubated in 0.3% Triton X-100 in DPBS for 30 minutes to permeabilize cell membranes, and blocked for one hour in goat serum dilution buffer (10% normal goat serum [Sigma-Aldrich], 0.3% Triton X-100 in DPBS). Monoclonal anti-synaptic vesicle protein-2 (SV2) antibody (1:30 dilution, Developmental Studies Hybridoma Bank, University of Iowa, Iowa City, Iowa) was applied overnight at 4° C. to visualize photoreceptor terminal and axon retraction into the ONL. Sections were rinsed with DPBS, incubated with blocking solution for one hour, and incubated in goat anti-mouse FITC secondary antibody (1:50 dilution, Jackson ImmunoResearch Laboratories, Inc., West Grove, Pa.) for one hour at room temperature. Sections were then washed with DPBS, and propidium iodide (1:100 dilution in DPBS; Sigma-Aldrich) was applied for one hour at room temperature to visualize the ONL. Omission of primary antibody constituted control immunolabelling. Sections were washed three times with DPBS, covered with antifade medium (Vectashield, Vector Laboratories, Burlingame, Calif.), coverslipped, and sealed with nail polish.

Optical sections were obtained with a laser confocal microscope (LSM 510; Carl Zeiss, Oberkochen, Germany) using a 40×/1.2 water immersion objective lens; 488 nm and 543 nm excitation filters were used for FITC (green) and propidium iodide (red) staining, respectively. Gain and amplitude were maintained throughout a single experiment in order to detect changes in labeling patterns between conditions. Photoreceptor axon and terminal retraction, indicated by the area of SV2 labeling in the ONL, and ONL thickness (i.e., width in cross section) and stratification (i.e., number of nuclei in cross section) were analyzed using Image J (version 1.42q; NIH imaging software; http://rsbweb.nih.gov/ij/). Three areas, 50-100 μm apart, were photographed per explant section, and three sections, approximately 350 μm apart, were photographed per explant. For SV2 evaluation, a standard threshold was assigned to all images of the same experiment to control for background fluorescence. The total area (pixels²) of fluorescein labeling was measured within an area outlining the ONL of each area photographed. A perpendicular line was drawn through the ONL to determine the width in cross-section and the number of nuclei in each cross section was determined. Morphometry was performed without knowledge of the experimental conditions (i.e., blinded).

Statistical Analysis

Statistical analysis was performed using Sigma Plot 11 (Systat Software Inc., San Jose, Calif.). Significance was accepted at p<0.05. Kruskal-Wallis One Way Analysis of Variance on Ranks followed by the Dunn's Method for Multiple Comparisons vs. Control group, and the Wilcoxon Signed Rank tests were used to compare the effects between media within and between time points, and between different media preparations. An unpaired t-test was used to compare trophic factor concentrations from this paper vs. those quantified previously (Kolomeyer A M, et. al. Invest Ophthalmol Vis Sci 2011; 52:5973-5986). All data are expressed as means with standard errors of the mean.

### Results

Trophic Factor Protein Secretion

RPE-CM neurotrophic secretion profile was quantified and compared to that reported in a previous study (Table 3). Of the seven growth factors tested, HB-EGF, HGF, VEGF, and PEDF were similar to that reported previously for RPE-CM prepared from fetal RPE of similar passage and time in culture (Kolomeyer A M, et. al. Invest Ophthalmol Vis Sci 2011; 52:5973-5986).

TABLE 20
Comparisons of trophic factor concentrations in RPE-CM with a previously
published study.

| | Mean ± SD (pg/μg RPE protein) | | | | | | |
|---|---|---|---|---|---|---|---|
| Study | HB-EGF | HGF | VEGF-A | NGF | BDNF | CNTF | PEDF |
| Current | 91.7 ± 26.6 | 309.6 ± 271.0 | 17496.5 ± 13246.1 | 24.2 ± 8.5 | 27.1 ± 17.3 | 261.7 ± 60.5 | 572472.1 ± 336019.1 |
| (n = 3) | | | | | | | |
| Previous | 149.0 ± 36.5 | 521.4 ± 126.4 | 19719.5 ± 3445.3 | 14.6 ± 7.6 | 321.9 ± 64.1 | 35.0 ± 9.4 | 803526.0 ± 242560.3 |
| (n = 5) | | | | | | | |
| P | >0.05 | >0.05 | >0.05 | 0.02* | 0.01* | 0.004* | >0.05 |

Trophic factor concentrations (pg/μg RPE protein) in 100% passage-2, day-7 fetal RPE conditioned media were quantified by multiplex ELISA and compared for statistical significance (p < 0.05) in the current vs. previously published study⁸ using an un-paired t-test.
*NGF and CNTF were significantly higher and BDNF was significantly lower vs. previously published study.

Preservation of Porcine Retina Following RPE-CM Treatment

Heating and Proteinase-K Digestion

Heating the original RPE-CM significantly decreased its ability to reduce retinal cytotoxicity at the 24- and 48-hour time points and apoptosis at all time points (i.e., 6, 24, and 48 hours); whereas proteinase-K treatment significantly reduced its ability to decrease retinal cytotoxicity and apoptosis at all time points. The combined, sequential method of heating followed by proteinase-K treatment was similar to each method singly (i.e., heating or proteinase-K treatment) at reducing the ability of the original RPE-CM to decrease retinal cytotoxicity at the 24- and 48-hour time points (FIG. 9A); however, the combined treatment was more effective than heating only at decreasing the anti-apoptotic effect of the original RPE-CM at the 6- and 24-hour time points (FIG. 9B). There were no statistically significant differences between the original vs. sepharose-treated original CM and the heated vs. sepharose, heat-treated CM.

FIG. 9 illustrates the effect of heating and proteinase-K treatment on RPE-CM modulation of porcine retinal cytotoxicity (FIG. 9A) and apoptosis (FIG. 9B). A. Porcine retina (n=3) was cultured for 6-48 hours in untreated, heated, proteinase-k digested, and heated plus proteinase-k digested 100% passage-2, day-7 fetal RPE-CM. Quantification of mean±SEM Lactate Dehydrogenase Concentration (FIG. 9A) and DNA Fragmentation (FIG. 9B) optical densities (at 490 nm and 405 nm-490 nm, respectively) were compared for statistical significance (p<0.05) by Kruskal-Wallis One Way Analysis of Variance on Ranks followed by the Dunn's Method for Multiple Comparisons vs. Control group.

RPE-CM Efficacy at Different Concentrations

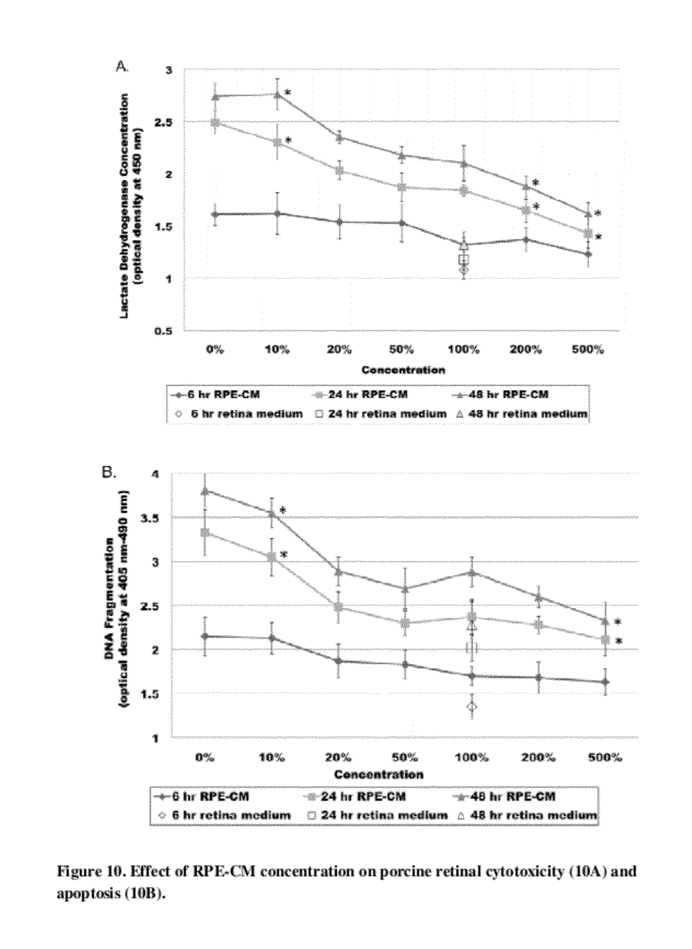

Retinal cytotoxicity studies show a significant decline in toxicity with increasing concentrations of RPE-CM at the 24- and 48-hour incubation periods (FIG. 10A). The 6-hour incubation period showed similar cytotoxicity to that of 100% RPE-CM for all concentrations. The concentrated RPE-CM (200% and 500%) were significantly better than 100% RPE-CM at 24- and 48-hour time points; however; neither was as effective as retina medium at these time points. Retinal apoptosis showed a decline with increasing RPE-CM concentration at all incubation periods for the 20% and higher concentrations; the 10% RPE-CM showed apoptosis values similar to DMEM (FIG. 10B). The 500% RPE-CM was statistically similar to that of retina medium at the 24- and 48-hour time points.

FIG. 10 illustrates the effect of RPE-CM concentration on porcine retinal cytotoxicity (FIG. 10A) and apoptosis (FIG. 10B). Values for 100% retina medium are included for comparison. Porcine retina (n=3) was cultured for 6-48 hours in DMEM (corresponding to 0%) and 10%, 20%, 50%, 100%, 200%, and 500% passage-2, day-7 fetal RPE-CM. Mean±SEM Lactate Dehydrogenase Concentration (FIG. 10A) and DNA Fragmentation (FIG. 10B) optical densities (at 490 nm and 405 nm-490 nm, respectively) were compared for statistical significance (p<0.05) by Kruskal-Wallis One Way Analysis of Variance on Ranks followed by the Dunn's Method for Multiple Comparisons vs. Control group. *p<0.05 vs. 100% RPE-CM.

Fractionation

In relation to unfiltered RPE-CM, at the 24- and 48-hour time points, the 3-100 kDa sub-fraction was not any different in the ability to decrease retinal cytotoxicity (p=0.14 and 0.09, respectively) or apoptosis (p=0.28 and 0.08, respectively).

Recombinant Proteins and Neutralizing Antibodies

Depending on the culture condition, there was a trend for significantly more porcine retinal cytotoxicity and apoptosis at the corresponding 48- vs. 24-hour time points (p-values ranged from 0.008-0.063 and 0.008-0.095, respectively). After 24 and 48 hours of culture, compared to DMEM, RPE-CM decreased retinal cytotoxicity by 21% and 17%, respectively, and apoptosis by 34% and 34%, respectively. This effect was significant (p<0.05) at all time points except for the 48-hour retinal cytotoxicity culture condition.

With respect to DMEM, when added singly, 50% PEDF, HGF, and VEGF-A receptor occupancies resulted in a 15% and 10%, 10% and 1.9%, and 6.6% and 4.1% decrease in retinal cytotoxicity, and a 16% and 7.1%, 6.6% and 6.0%, and 7.0% and 4.5% decrease in apoptosis after 24 and 48 hours of culture, respectively, but the differences were not statistically significant (Table 21; p>0.05). Compared to DMEM, when added singly, 90% PEDF, HGF, and VEGF-A receptor occupancies resulted in a 14% and 24%, 15% and 18%, and 2.5% and 0% decrease in retinal cytotoxicity, and a 22% and 22%, 21% and 20%, and 12% and 6.9% decrease in apoptosis at the 24- and 48-hour time points, respectively. The reductions in retinal cytotoxicity and apoptosis for 90% PEDF and 90% HGF as compared to DMEM were significant at all time points (Table 4; p<0.05).

TABLE 21
Porcine retina viability after culture
in DMEM with recombinant proteins.

| | Mean ± SEM (%) relative to DMEM | | | |
|---|---|---|---|---|
| | Lactate | | DNA | |
| DMEM | Dehydrogenase | | fragmentation | |
| culture medium | 24 hr | 48 hr | 24 hr | 48 hr |
| +50% PEDF | 84.9 ± 3.1 | 89.6 ± 6.2 | 83.8 ± 6.4 | 92.3 ± 4.4 |
| saturation | | | | |
| (n = 5) | | | | |
| +50% HGF | 89.7 ± 3.9 | 98.1 ± 2.8 | 93.4 ± 7.2 | 94.0 ± 5.9 |
| saturation | | | | |
| (n = 5) | | | | |
| +50% VEGF-A | 93.4 ± 6.4 | 95.9 ± 6.0 | 93.0 ± 3.6 | 95.5 ± 2.6 |
| saturation | | | | |
| (n = 5) | | | | |
| +90% PEDF | 85.7 ± 4.4* | 75.8 ± 6.2* | 78.4 ± 3.6* | 77.7 ± 4.8* |
| saturation | | | | |
| (n = 9) | | | | |
| +90% HGF | 85.1 ± 3.6* | 82.2 ± 5.2* | 79.0 ± 3.9* | 80.1 ± 4.3* |
| saturation | | | | |
| (n = 9) | | | | |
| +90% VEGF-A | 97.5 ± 9.0 | 101.0 ± 5.8 | 87.9 ± 5.8 | 93.1 ± 4.4 |
| saturation | | | | |
| (n = 5) | | | | |

Porcine retina was cultured in different preparations of DMEM + recombinant proteins for up to 48 hours. The Kruskal-Wallis One Way Analysis of Variance on Ranks followed by the Dunn's Method for Multiple Comparisons vs. negative control (DMEM) was used to assess stastically signfiicant differences between the degree of retinal cytotoxicity and apoptosis at the 24- and 48-hour time points for the three culture conditions.
*p < 0.05 vs. DMEM.

Addition of PEDF antibody to RPE-CM decreased its ability to reduce retinal cytotoxicity by 11% and 7.5%, and apoptosis by 27% and 22.9% at the 24- and 48-hour time points, respectively (Table 5; p<0.05 for retinal apoptosis at both time points). Adding HGF antibody to RPE-CM decreased its ability to reduce retinal cytotoxicity by 5.9% and 4.9%, and apoptosis by 3.5% and 5.2% at the 24- and 48-hour time points, respectively (Table 22; p>0.05 at all time points). However, treating RPE-CM with a combination of PEDF and HGF antibodies reduced its ability to decrease retinal cytotoxicity by 19% and 15%, and apoptosis by 25% and 21% at the 24- and 48-hour time points, respectively (Table 5; p<0.05 at all time points). These results were not due to non-specific antibody binding since addition of IgG control to RPE-CM did not significantly reduce its ability to decrease retinal cytotoxicity and apoptosis after 24 and 48 hours of culture.

TABLE 22
Porcine retina viability after culture in RPE-CM with neutralizing antibodies.

| | Mean ± SEM (%) relative to RPE-CM | | | |
|---|---|---|---|---|
| RPE-CM culture medium | Lactate Dehydrogenase | | DNA fragmentation | |
| (n = 7) | 24 hr | 48 hr | 24 hr | 48 hr |
| +PEDF antibody | 111.4 ± 10.9 | 107.5 ± 8.6 | 127.0 ± 11.8* | 122.9 ± 7.5* |
| +HGF antibody | 105.9 ± 8.5 | 104.9 ± 7.9 | 103.5 ± 5.8 | 105.2 ± 7.0 |
| +PEDF and HGF antibodies | 118.8 ± 9.2* | 115.1 ± 6.1* | 124.7 ± 8.3* | 121.1 ± 11.1* |

Porcine retina was cultured in different preparations of RPE-CM + neutralizing antibodies for up to 48 hours. The Kruskal-Wallis One Way Analysis of Variance on Ranks followed by the Dunn's Method for Multiple Comparisons vs. positive control (RPE-CM) was used to assess stastically signfiicant differences between the degree of retinal cytotoxicity and apoptosis at the 24- and 48-hour time points for the three culture conditions.
*p < 0.05 vs. RPE-CM.

Preservation of Aged Human Retina

RPE-CM was significantly better (p<0.05) than DMEM at reducing retinal cytotoxicity in non-AMD and AMD retina after 24 and 48 hours of culture, and in African American retina at 24 hours only. It was also significantly better (p<0.05) than DMEM at reducing retinal apoptosis in all retinas at both time point (Table 23). No significant differences in retinal cytotoxicity and apoptosis with respect to ethnicity and ocular pathology were observed (p=0.31 and 0.34, respectively).

TABLE 23
Effect of RPE-CM on human retina viability.

| | | Mean ± SEM (optical density) | | | |
|---|---|---|---|---|---|
| | | Retinal Cytotoxicity | | Retinal Apoptosis | |
| Type of | | (LDH Concentration) | | (DNA Fragmentation) | |
| retina | Medium | 24-hour | 48-hour | 24-hour | 48-hour |
| Non-AMD | Retina medium | 0.88 ± 0.04* | 1.04 ± 0.04* | 0.91 ± 0.07* | 1.10 ± 0.09* |
| Caucasian | DMEM | 1.39 ± 0.04 | 1.56 ± 0.07 | 1.58 ± 0.09 | 2.05 ± 0.11 |
| | RPE-CM | 1.18 ± 0.05^{†} | 1.31 ± 0.08^{†} | 1.15 ± 0.08^{†} | 1.52 ± 0.11^{†} |
| AMD | Retina medium | 0.83 ± 0.03* | 0.91 ± 0.04* | 1.50 ± 0.12* | 1.78 ± 0.08* |
| Caucasian | DMEM | 1.27 ± 0.06 | 1.53 ± 0.06 | 2.30 ± 0.16 | 2.66 ± 0.16 |
| | RPE-CM | 1.06 ± 0.06^{†} | 1.27 ± 0.05^{†} | 1.89 ± 0.14^{†} | 2.23 ± 0.09^{†} |
| African | Retina medium | 0.90 ± 0.04* | 0.94 ± 0.05* | 1.26 ± 0.09* | 1.42 ± 0.07* |
| American | DMEM | 1.40 ± 0.07 | 1.46 ± 0.09 | 1.98 ± 0.12 | 2.21 ± 0.12 |
| | RPE-CM | 1.14 ± 0.07^{†} | 1.25 ± 0.09 | 1.57 ± 0.10^{†} | 1.72 ± 0.10^{†} |

Retina from non-AMD and AMD Caucasian as well as African American donors was cultured in retina medium, DMEM, and 100% RPE-CM. The degree of retinal cytotoxicity and apoptosis at the 24- and 48-hour time points was compared between the three culture conditions for statistical significance (p < 0.05) using the Kruskal-Wallis One Way Analysis of Variance on Ranks followed by the Dunn's Method for Multiple Comparisons vs. Control group.
*Significantly (p < 0.05) better than RPE-CM and DMEM.
^{†}Significantly (p < 0.05) better than DMEM.

Porcine Retinal Morphology

Compared to time 0 controls, all culture conditions had significantly decreased ONL width and decreased numbers of nuclei in cross-section as well as significantly increased amounts of photoreceptor terminal/axon retraction as measured by SV2 labeling in the ONL (Table 24). ONL width in cross-section was maintained in all media between the 24 and 48 hour time points. The preservation of ONL width in cross-section was significantly less in DMEM than in retina medium or RPE-CM at both time points. ONL stratification, as measured by the number of ONL nuclei in cross section, was maintained in media between the 24 and 48 hour time points. ONL stratification was significantly lower in DMEM than in retina medium and RPE-CM within each time point. SV2 labeling in the ONL was similar in the two time points for retina medium and DMEM; RPE-CM showed significantly more SV2 labeling at 48 hours compared to 24 hours. DMEM SV2 labeling was significantly higher than that observed in retina medium and RPE-CM within each time point.

TABLE 24
Porcine retina morphology after incubation in RPE-CM.

| | Mean ± SEM | | |
|---|---|---|---|
| | ONL width in | No. of ONL nuclei | SV2 (pixels²) in |
| Condition/treatment | cross section (μm) | in cross section | ONL |
| Time 0 (n = 4) | 131.4 ± 6.6* | 6.3 ± 0.2* | 66.2 ± 11.8* |
| 24-hour retina medium (n = 3) | 121.8 ± 6.9 | 5.8 ± 0.3 | 338.1 ± 34.6 |
| 24-hour DMEM (n = 3) | 94.3 ± 4.1^{†} | 4.8 ± 0.2^{†} | 561.6 ± 58.5^{†} |
| 24-hour RPE-CM (n = 9) | 113.8 ± 2.5^{#} | 5.5 ± 0.2^{#} | 394.4 ± 31.6 |
| 48-hour retina medium (n = 3) | 122.3 ± 5.7 | 5.9 ± 0.2 | 361.3 ± 27.9 |
| 48-hour DMEM (n = 3) | 95.7 ± 5.2^{†} | 4.8 ± 0.2^{†} | 589.8 ± 67.3^{†} |
| 48-hour RPE-CM (n = 9) | 111.5 ± 3.0^{#} | 5.5 ± 0.2^{#} | 472.0 ± 17.6^{#§} |

Porcine retina was cultured in retina medium, DMEM, and 100% RPE-CM for 24 and 48 hours. At time 0 and at 24 and 48 hours after the start of the experiment, porcine retina morphologic parameters including ONL width and the number of nuclei in cross-section as well as the amount of SV2 in the ONL were quantified. The data were compared in the following ways: 1) time 0 vs. 24- and 48-hour time points; 2) retina medium vs. DMEM vs. RPE-CM at the 24- and 48-hour time points; and 3) 24- vs. 48-hour time point for each corresponding culture condition.
*Significantly (p < 0.05) better than RPE-CM and DMEM at both time points.
^{†}Significantly (p < 0.05) worse than retina medium and RPE-CM.
^{#}Significantly (p < 0.05) worse than retina medium.
^{§}Significantly (p < 0.05) worse than 24-hour RPE-CM.
Abbreviations: ONL, outer nuclear layer; SV2, synaptic vesicle protein-2.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications U.S. patents, references and GenBank sequences cited in this disclosure are incorporated by reference in their entireties. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

1. A pharmaceutical composition comprising a pharmaceutically acceptable carrier and at least two biologically active trophic factors selected from the group consisting of LIF, bFGF, HB-EGF, VEGF-A, NGF, BDNF, CNTF, PEDF, IGFBP-3, isoform 1 of semaphoring-3B, TGF-β and HGF.

2. The pharmaceutical composition of claim 1 wherein, the selected biologically active trophic factors are HGF and PEDF.

3. The pharmaceutical composition of claim 1 comprising LIF, bFGF, HB-EGF, VEGF-A, NGF, BDNF, CNTF, HGF and PEDF.

4. The pharmaceutical composition of claim 1, wherein the pharmaceutically acceptable carrier is capable of sustained release delivery of said factors.

5. A method to decrease the degeneration of a retina associated with an ocular condition in a subject in need thereof comprising administration to said subject an effective amount of the pharmaceutical composition of claim 1.

6. The method of claim 5, wherein the pharmaceutical composition is administered by intraocular administration.

7. The method of claim 5, wherein said ocular condition is selected from the group consisting of age-related macular degeneration, retinitis pigmentosa, glaucoma, optic atrophy, ocular inflammation, retinopathy, diabetic retinopathy, retinal ganglion cell dysfunction, and retinal detachment.

8. A method to select a group of cells that secrete factors that decrease the degeneration of a retina comprising:
a. isolating medium from a group of cultured cells;
b. culturing a degenerating animal retina with said isolated medium;
c. determining the degeneration of the retina; and
d. comparing the level of the degeneration of the retina to a standard;
wherein the group of cells are selected if said isolated medium decreased the degeneration of the retina compared to the standard.

9. The method of claim 8, where in the group of cells are stem cells capable of differentiating to retinal pigment epithelial cells, or fetal retinal pigment epithelial cells.

10. The method of claim 8, wherein the determining step is conducted by performing a cytotoxicity assay and/or an apoptotic assay.

11. The method of claim 8, wherein the standard is the level of the degeneration of a retina cultured in non-conditioned medium.

12. The method of claim 8, wherein the cells are human cells

13. The method of claim 8, wherein the group of cultured cells is cultured in a solid support.

14. The method of claim 8, wherein the degenerating retina is cultured in a solid support.

15. The method of claim 8, wherein the animal retina is a human retina or a porcine retina.

16. Conditioned medium isolated from fetal retinal pigment epithelial cells cultured in medium in a solid support, wherein said medium is isolated after said cells have been passaged from 2 to 6 passages and within 7 days of said passage.

17. A method of identifying cells for transplantation to a subject having an ocular condition comprising:
a. isolating medium from a group of cultured cells;
b. culturing a degenerating animal retina with said isolated medium;
c. determining the degeneration of the retina; and
d. comparing the level of the degeneration of the retina to a standard;
wherein the group of cells are selected if said isolated medium decreased the degeneration of the retina compared to a standard.

18. The method of claim 17, wherein said group of cells are stem cells isolated from the subject.

19. The method of claim 17, wherein the cells are human cells

20. The method of claim 17 wherein the group of cultured cells is cultured in a solid support.

21. The method of claim 17 wherein the degenerating retina is cultured in a solid support.

22. The method of claim 17 wherein the animal retina is a human retina or a porcine retina.

23. A kit for identifying an agent that decreases degeneration of a retina wherein the kit comprises an explant culture of full thickness animal retina.

24. The kit of claim 23 wherein the retina is a human or a porcine retina.

25. The kit of claim 23 further comprising reagents for performing an assay for measuring retinal degradation.
